(19) United States
(12) Patent Application Publication
(10) Pub. No.: US 2023/0014385 A1
(43) Pub. Date: Jan. 19, 2023

(54) SYSTEMS AND METHODS OF DETERMINING NUMBER OF POSTURE CHANGES FOR A GROUP AND DETERMINING OPTIMAL OPERATING MODELS FOR INTELLIGENT AUTOMATED CHAIRS

(71) Applicant: Movably, Inc., Lexington, MA (US)

(72) Inventor: Mark Kapij, Lexington, MA (US)

(73) Assignee: Movably, Inc., Lexington, MA (US)

(21) Appl. No.: 17/865,550

(22) Filed: Jul. 15, 2022

Related U.S. Application Data
(60) Provisional application No. 63/221,964, filed on Jul. 15, 2021.

(51) Int. Cl. A47C 7/02; A47C 7/00; A61B 5/11; A61B 5/00

(52) U.S. Cl. CPC A47C 7/024; A47C 7/004; A61B 5/1116; A61B 5/6891; A47C 9/025

(57) ABSTRACT

The present disclosure is directed to a method of collecting and using data obtained from an intelligent automated chair to optimize workflow and increase a user or group's health and productivity. Sensors in the intelligent automated chair can include motion, touch, heart rate, weight, presence, sound, keystroke, etc. In addition, posture, health, and productivity data are collected and compared over periods of time along with a learning algorithm to recommend specific operating models to increase a user or group's overall posture, health, and productivity. The methods and systems are configured to recommend action steps based on usage that can include a chair training program, nutritional training program, mental health training, bonus consideration, rewards program, advancement consideration, etc. Furthermore, the metrics and recommendations can assist with addressing issues associated with absenteeism and presenteeism. Lastly, data collected can be used to create forecasting models based on productivity and health cost.

### CROSS-REFERENCE TO RELATED APPLICATIONS

This application claims the benefit of U.S. Provisional Patent Application No. 63/221,964 filed on Jul. 15, 2021, which is herein incorporated by reference in its entirety.

### TECHNICAL FIELD

The present disclosure generally relates to the field of determining human movement, and more particularly to posture changes during a work shift.

### BACKGROUND

Office furniture has historically been designed to provide comfort for working hours on end. However, being in one position for extended periods of time can have negative impacts on your health. One such article that documents the need to switch positions frequently is Rethinking design parameters in the search for optimal dynamic seating by Jennifer Pynt, PhD, Grad Dip Manip Ther, Dip Physio published by the Journal of Bodywork & Movement Therapies (2015) 19, 291-303. In this particular article, it illustrates the negative effects of 10-20 minutes of sustained slouched sitting. Similarly, standing too long in the same position can also have negative impacts.

Apps have been used to try to remind individuals to get up and move, and sometimes utilize step counters or other sensors attached to smartphones or smartwatches to determine how long it has been since the last time a person stood up or took a certain number of steps within a time period. However, there is no technology to tracking posture changes, and in particular posture changes amongst a group of individuals, which information could help quantify the overall health of a group. The present application seeks to provide this and other solutions that will be apparent to those in the art.

### SUMMARY OF THE INVENTION

Described herein are systems and methods that identify the number of posture changes for individuals and groups over a period of time using an intelligent automated chair. In some variations, the tracked posture changes during a tracked period of time can be compared with health and/or productivity data over the same tracked period as well as with a control or second period of time, which second period of time could be before or after the tracked period of time associated with the tracked posture changes using intelligent automated chairs. The tracked posture change data and health or productivity data can be used to generate forecasting models as well as used to alter operational models of a portion of the intelligent automated chairs to impact health or productivity measurements. The tracked data (posture, health and productivity) can also be used to generate recommended action steps for at least a sub-set or portion of users of a group of users using the intelligent automated chairs.

For example in one embodiment a method creating an operating model for a plurality of intelligent automated chairs comprising the steps of: tracking posture change data associated with a plurality of intelligent automated chairs for a first period of time; tracking health or productivity data over the first period of time; tracking health or productivity data over a second period of time; comparing the tracked health or productivity data over the first period of time with the second period of time; generating an operating model for at least some of the plurality of intelligent automated chairs based on the compared health or productivity data.

In another embodiment a method of determining the number of posture changes for a group over a period of time comprises the steps of: 1) providing in a given area a plurality of automated chairs, each automated chair comprising: i) a base portion, ii) a vertical support extending from the base portion, iii) a horizontal support interfacing with the vertical support, iv) a right leaf extending from the horizontal support and configured to be driven by a right motor that causes the right leaf to alter between positions of horizontal and vertical, v) a left leaf extending from the horizontal support and configured to be driven by a left motor that causes the left leaf to alter between positions of horizontal and vertical, and vi) an automated control assembly electrically coupled to the right motor and the left motor, and configured to operate according to an automated shifting pattern that causes the right leaf and left leaf to change positions, wherein each position change is associated with a posture change, and wherein the automated control assembly is also configured to receive user input that can modify the automated shifting pattern; 2) tracking a number of posture changes associated with each of the plurality of automated chairs over the period of time based on the position changes; and 3) receiving into a database the tracked number of posture changes associated with each of the plurality of automated chairs over the period of time.

The above method can further include the step of tracking over the same period of time a productivity measurement. The productivity measurement can include one of the of the following: sales, customer support metrics, periodic reviews, periodic ratings, project completion timelines, work quality, attrition rate, and production output. Additionally, this above method can further include the step of comparing the productivity measurement to the tracked number of posture changes. Furthermore, this above method can further include the step of recommending a modification to the number of posture changes to be achieved by the group over a new period of time.

The above method can alternatively further include the step of tracking over the same period of time a health measurement. The health measurement includes one of the of the following: number of sick days, costs spent on medical care, types of medical care needed, number of health claims, number of work-related injuries, type of work-related injuries, costs spent on prescriptions, blood test measures, heart rate, body weight, amount of coffee consumed, and amount of water consumed. Additionally, this above method can further include the step of comparing the health measurement to the tracked posture change. Furthermore, this above method can further include the step of recommending a modification to the number of posture changes to be achieved by the group over a new period of time.

Either of the above variations to the above method can further include the step of updating the automated shifting pattern for at least a subset of the automated chairs based on the recommended modification to the number of posture changes to be achieved by the group over the new period of time.

In yet another variation to the above method, an intelligent analysis module associated with the plurality of automated chairs can be configured to update the automated shifting pattern for at least a subset of automated chairs based on health data or productivity data associated with the group from a control period of time compared to health data or productivity data associated with the group from a period of time.

The automated chairs can include one or more sensors, and wherein the one or more sensors are configured to detect data that that is used to determine a productivity or health measurement. Examples being a heart rate measuring sensor, a weight sensor, a presence detection sensor, a keystroke detection module, and so forth.

Alternatively, the intelligent analysis module can perform the step of: parsing the group into at least two sub-groups based on the tracked number of posture changes associated with each of the plurality of automated chairs. This can help implement a further step of recommending an action step for at least one of the sub-groups based on tracked posture changes associated with the at least one subgroup. The action step can be to consider providing one of the following: automated chair training program, nutritional training program, mental health training program, free medical treatment program, bonus consideration, rewards program, advancement consideration, skills training program, and increased vacation program.

In yet another variation to the above method, it can further include the step of: generating a productivity forecast, using a forecasting analysis module, based on the tracked number of posture changes associated with each of the plurality of automated chairs over the period of time and at least one measured productivity measurement over the period of time. The forecasting analysis module can be run on a cloud-based system.

Similar to the above, a step of generating a health cost forecast, using a forecasting analysis module, based in part on the tracked number of posture changes associated with each of the plurality of automated chairs over the period of time and at least one measured health measurement over the period of time can be another variation.

The automated shifting pattern in the above method and variations can include at least one of the following parameters: sequences of positions, durations between each position change, varying durations throughout the day, and varying durations specific to each position.

The above method can also include combining the steps of: tracking over the same period of time a productivity measurement; tracking over the same period of time a health measurement; and generating an updated automated shifting pattern based on the tracked productivity and health measurements. These combination steps can further include the step of generating a health costs or productivity forecast based on the updated automated shifting pattern.

In yet another variation to the above method of determining the number of posture changes for a group over a period of time, the step of: tracking health measurement or productivity measurement data along with tracked posture data over several periods of time, wherein the automated shifting pattern is modified between each period to target a different range of posture changes; and determining an optimized range of posture changes for the group can be included. Adding to these can include the step of: implementing into an optimization operation model that guides the automated shifting pattern in the determined optimized range of posture changes.

For any of the above a variations, the user can modify the automated shifting pattern in at least one of the following of manners: altering the frequency of the posture changes, altering the type of posture changes used, altering the posture change pattern, pausing the automated chair from continuing through the current automated shifting pattern, altering the duration of time between changes for one or more posture positions, altering the duration of time between changes for one or more periods of time.

In yet another embodiment a method of determining the number of posture changes for a group over a period of time comprises the steps of: providing in a given area a plurality of automated chairs, each automated chair comprising: a base portion; a vertical support extending from the base portion; a horizontal support interfacing the vertical support; a right leaf, configured to be driven by a motor in response to an input to alter between positions of horizontal and vertical; a left leaf, configured to be driven by a motor in response to an input to alter between positions of horizontal and vertical; and an automated control assembly electrically coupled to the right leaf and the left leaf, and configured to receive input data from one or more sensors, create an adjustment to at least one portion of the automated chair based on the received input data, and track the posture changes associated with the use of the automated chair over the period of time; and receiving into a database the tracked posture changes from each of the plurality of automated chairs over the period of time.

One system embodiment of an intelligent automated chair system comprises: an automated chair comprising: a base portion; a vertical support extending from the base portion; a horizontal support interfacing the vertical support; a right leaf, configured to be driven by a right motor in response to an input to alter between positions of horizontal and vertical; a left leaf, configured to be driven by a motor in response to an input to alter between positions of horizontal and vertical; and an automated control assembly electrically coupled to the right leaf and the left leaf, and configured to receive an updatable operating model based on compared tracked health or productivity data from a first period of time and a second period of time, wherein during the first period of time the automated chair was not used and during the second period of time the automated chair was used.

In another embodiment, a method for creating an operating model for a plurality of intelligent automated chairs comprising the steps of: 1) tracking posture change data associated with a plurality of intelligent automated chairs for a first period of time; 2) tracking health or productivity data over the first period of time; 3) tracking health or productivity data over a second period of time; 4) comparing the tracked health or productivity data over the first period of time with the second period of time; 5) generating an operating model for at least some of the plurality of intelligent automated chairs based on the compared health or productivity data.

This method for creating an operating model for a plurality of intelligent automated chairs can include intelligent automated chairs that are comprised of: a base portion, a vertical support extending from the base portion, a horizontal support interfacing with the vertical support, a right leaf extending from the horizontal support and configured to be driven by a right motor that causes the right leaf to alter between positions of horizontal and vertical, a left leaf extending from the horizontal support and configured to be driven by a left motor that causes the left leaf to alter between positions of horizontal and vertical, and an automated control assembly electrically coupled to the right motor and the left motor, and configured to operate according to the operating model.

The plurality of automated chairs can be configured to track the posture changes and relay the tracked data to a tracked database.

In yet another embodiment, a method for validating an operation model for use with an intelligent automated chair, wherein the intelligent automated chair includes: a base portion, a vertical support extending from the base portion, a horizontal support interfacing with the vertical support, a right leaf extending from the horizontal support and configured to be driven by a right motor that causes the right leaf to alter between positions of horizontal and vertical, a left leaf extending from the horizontal support and configured to be driven by a left motor that causes the left leaf to alter between positions of horizontal and vertical, and an automated control assembly electrically coupled to the right motor and the left motor, and configured to operate according to the operation model comprises the steps of: 1) tracking usage information associated with the intelligent automated chair utilizing the operation model over a first period of time; 2) tracking data for a plurality of health measurements or productivity measurements over the first period of time; 3) tracking data for the plurality of health measurements or productivity measurements over a second period of time, wherein the intelligent automated chair is not be utilized; and 4) comparing the tracked data of the first period of time to the second period of time.

This method can further include the step of: 5) altering the operation model based on the compared tracked data from the first and second periods of time; 6) tracking data for the plurality of health measurements or productivity measurements over a third period of time, wherein the intelligent automated chair is operating utilizing the altered operational model; 7) comparing the tracked data from the third period of time with the tracked data from the first period of time or second period of time; and 8) determining if the operation model or altered operational model improved at least one of the tracked plurality of health measurements or tracked plurality of productivity measurements.

The tracked usage information can include any of the following: chair position changes, duration of certain positions, and number of paused or interrupted position changes.

The first period of time can be later than the second period of time in either of the above variations.

In yet another embodiment, a method for billing a customer using a plurality of intelligent automated chairs, wherein each intelligent automated chair includes: a base portion, a vertical support extending from the base portion, a horizontal support interfacing with the vertical support, a right leaf extending from the horizontal support and configured to be driven by a right motor that causes the right leaf to alter between positions of horizontal and vertical, a left leaf extending from the horizontal support and configured to be driven by a left motor that causes the left leaf to alter between positions of horizontal and vertical, and an automated control assembly electrically coupled to the right motor and the left motor, and configured to operate according to the operation model comprising the steps of: 1) tracking usage information associated with each intelligent automated chair over a first period of time; 2) determining if usage associated with any of the plurality of intelligent automated chairs has fallen below a usage threshold; and 3) billing the customer based on the usage of the intelligent automated chairs above the usage threshold.

The usage threshold can be comprised of a percentage of posture changes performed over a period of time by a user utilizing the intelligent automated chair. The usage threshold can also be comprised of the total number of posture changes performed over a period of time by a user utilizing the intelligent automated chair.

### BRIEF DESCRIPTION OF THE DRAWINGS

The foregoing and other objects, features, and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention, wherein:

FIGS. 1A-G illustrate various views of an embodiment of an intelligent automated chair.

FIGS. 2A-B illustrate various views of another embodiment of an intelligent automated chair.

FIGS. 3A-B illustrate various views of another embodiment of an intelligent automated chair with arm rests.

FIGS. 4A-C illustrate various views of the base portion of an intelligent automated chair including electrical power and wheels.

FIGS. 5A-D illustrate a change in position of an intelligent automated chair and the various configurations or positions a user could utilize on the intelligent automated chair.

FIGS. 6A-F illustrates various configurations of a version of an intelligent automated chair without a backrest.

FIGS. 7A-C illustrate various views of the automated control assembly used in the intelligent automated chairs.

FIG. 8 illustrates a schematic of a system of an intelligent automated chair using various forms of input to determine the operation of the intelligent automated chair.

FIG. 9 illustrates a workflow indicative of at least one mode of operation determination.

FIG. 10 illustrates a schematic where various input and decisions can occur in the intelligent automated chair system.

FIG. 11 illustrates one mode of operating the intelligent automated chair.

FIG. 12 illustrates a workflow for creating an operating mode and the parameters associated with the operating mode.

FIG. 13 illustrates a flowchart where the user receives a notification prior to the intelligent automated chair changing positions.

FIG. 14 is a flowchart that illustrates the interruptible nature of the intelligent automated chair.

FIG. 15 illustrates a flowchart of notifying the user or interrupting the operating mode based on sensed posture information.

FIGS. 16A-B illustrate various workflows for determining the number of posture changes for a group and providing one or more recommended posture change operating models.

FIGS. 16C-D illustrate various workflows for determining the number of posture changes for a group and providing one or more health or productivity forecasts.

FIG. 17 illustrates a workflow for determining an optimization range for the number of posture changes for a group.

FIG. 18 illustrates a workflow for parsing a group of intelligent automated chair users in subgroups and recommending an action step to at least one of the subgroups.

### DETAILED DESCRIPTION OF THE INVENTION

As noted in the background one of the problems that the present application is seeking to address is to minimize disrupting a person's work, while introducing an optimal amount of activity in the person to provide health benefits. Some of the health benefits for example can include slightly increasing the heart rate with some motion, which can allow the spinal disks to get nutrition via diffusion. By shifting positions periodically muscle fatigue and strain on various parts of the body are reduced. A slight increase in blood flow can also help with increasing oxygen to the brain, which can help with focus and concentration, which is often needed when performing various tasks at a desk, such as coding, legal work, accounting, engineering work, and so forth.

One of the proposed solutions to the problem described above involves automatically tracking posture changes for one or more users using an intelligent automated chair running an operational model that runs an automated shifting program. This operational model can be altered or updated based on various tracked data including health and productivity measurements.

The first portion of the description will focus on the intelligent automated chair embodiments that can then be implemented into the group environment and be part of the larger solution of increasing productivity, increasing health benefits, reducing health costs, forecasting health and productivity measures, and recommending actionable steps for at least one subset group of an overall group of users utilizing the intelligent automated chairs.

For purposes of this description additional description to certain terms is provided that include information in addition to those terms ordinary meaning to provide clarity.

Biosensor or biosensor feedback includes information associated with a user's health, body, or interactions with a user's body, which has been received by a sensing device or system configured to detect or determine such information. Examples include heart rate information, SpO2 levels, calories burned, weight of a user, number of steps the user has taken, skin pH levels, levels of compounds or minerals in blood, sweat, or urine, exposure to sunlight, hours slept including the various types of sleep cycles the user experienced, and so forth. These examples are meant to be exemplary and not limited.

Sensors include any device or mechanism that is configured to detect anything and can be inclusive of biosensors. Additional sensed examples could include the presence of a user, load on each of the right or left leaves of the chair, pressure or weight sensors in the base or foot rest of the chair, and so forth. These may or may not be directly associated with a user's health. The presence of a user can be performed by IR detection, Bluetooth proximity detection with a user's smartphone, weight detection by stepping onto the base of the chair and so forth. This presence detection is not necessarily associated with a user's health, but can indicate that the user is in proximity to the chair and begin operating according to an operating model associated with that user.

The term cloud refers to one or more computing devices, such as servers, that are generally located remotely from the user or intelligent automated chair. The cloud can be used to run algorithms and pattern detection models, to generate and recommend the appropriate operating model for a particular user.

Mobile computing device can include smartphones, tablets, laptops and even smartwatches that have wireless communication means, some processing capabilities configured to run an application and memory.

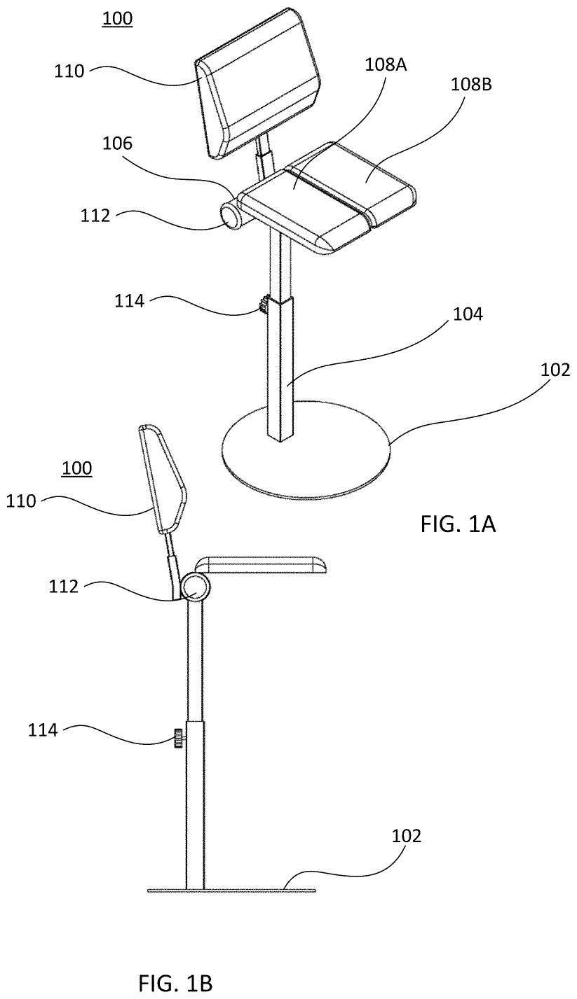

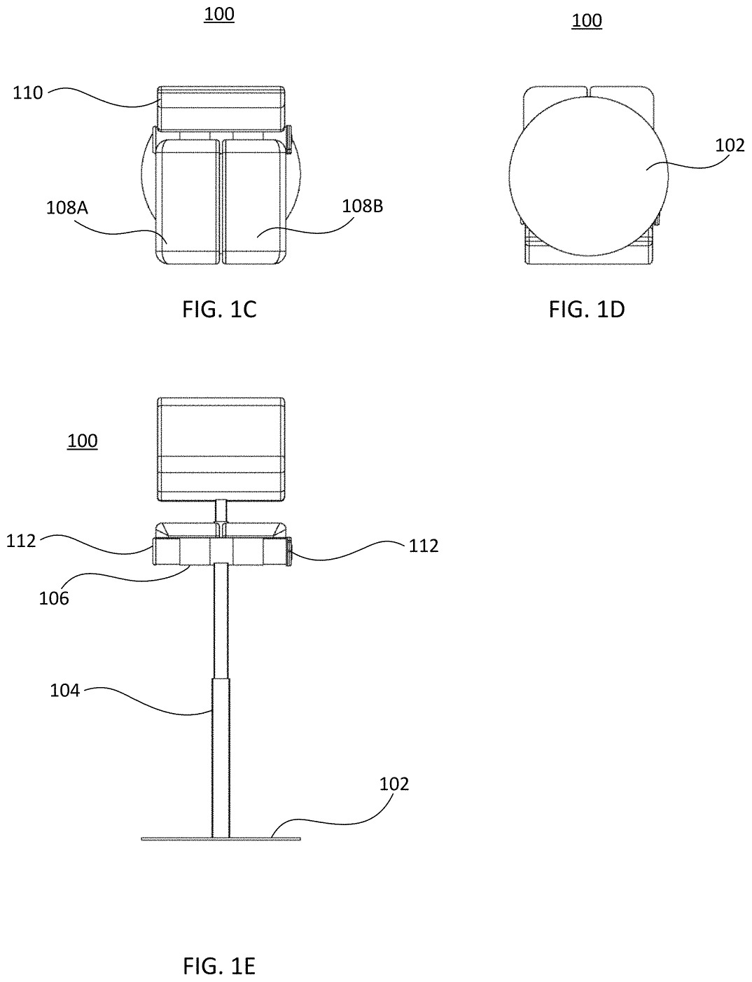

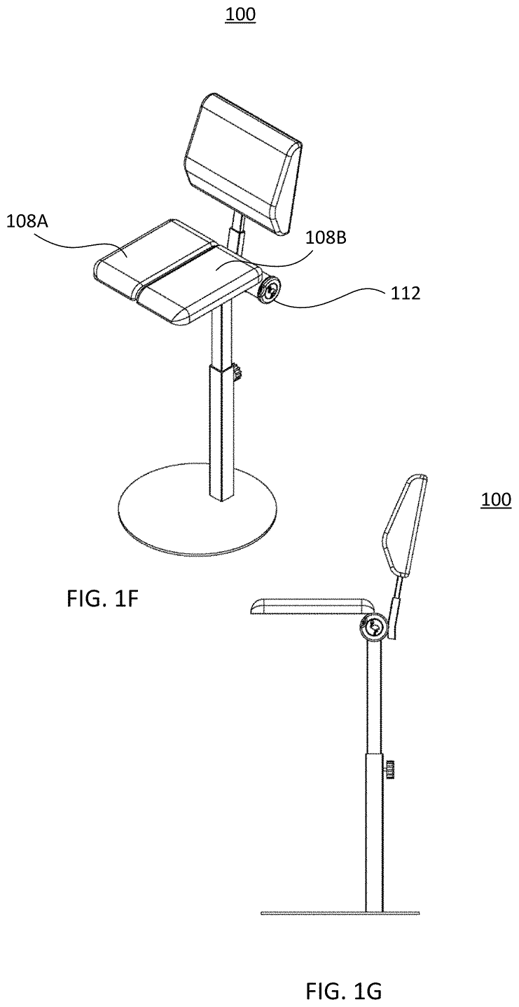

Several embodiments of intelligent automated chairs are disclosed herein. In one embodiment shown FIGS. 1A-G an intelligent automated chair 100 includes a base 102, a vertical support 104, and a horizontal support 106, which is configured to house a motor and control systems. The motor and control systems are configured to operate and alternate the right leaf 108A and left leaf 108B in various positions that will be shown in other embodiments below. A control interface 112 is disposed on either side of the horizontal support 106. Extending from the vertical support or alternatively from the horizontal support in some versions is a backrest 110. The vertical support can include a height adjustment mechanism 114. As shown in this embodiment 100, the height adjustment mechanism 114 is a manual adjustment, but in other versions this can be an automated adjustment.

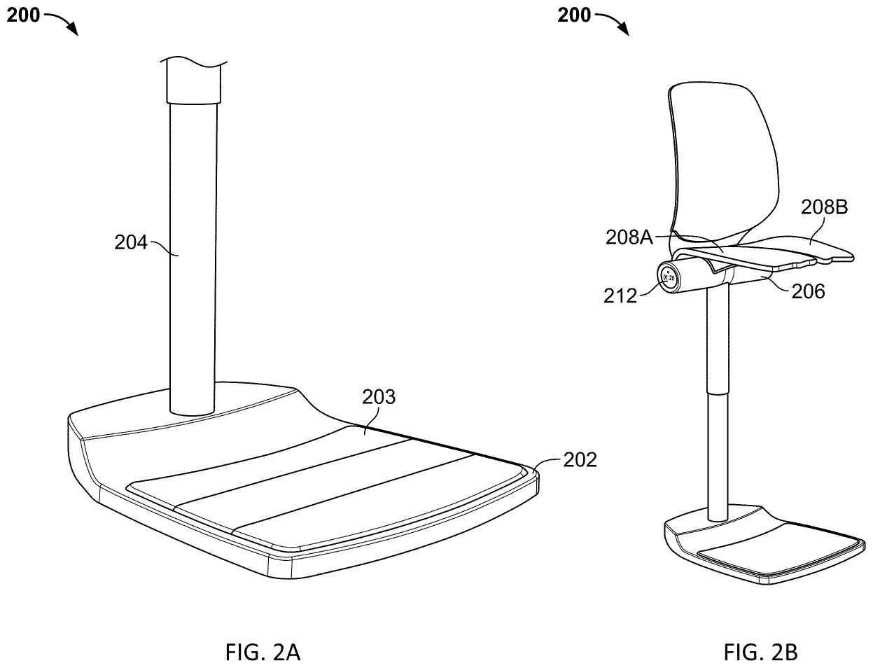

FIGS. 2A-B illustrate another embodiment of an intelligent automated chair 200, which includes a base 202, attached to a vertical support 204. Base 202 has a textured surface 203 configured to have a layer of cushion and support when a user is in the standing position. The textured surface 203 is also configured to be a non-slip surface in certain embodiments. Similar to embodiment 100, 200 also includes a control interface 212 on horizontal support 206, which is connected to right leaf 208A and left leaf 208B.

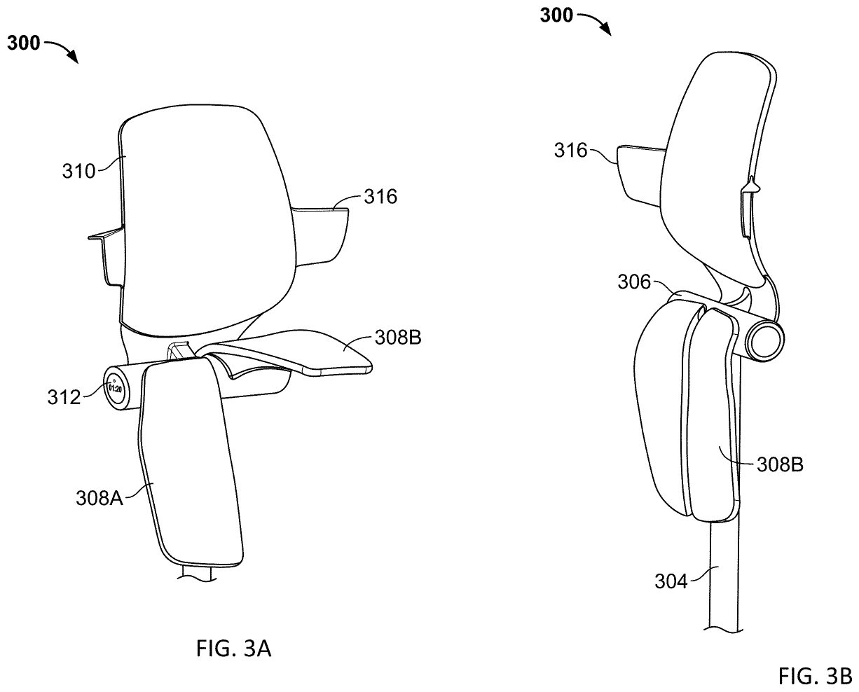

FIGS. 3A-B illustrate another embodiment of an intelligent automated chair 300, illustrating version that includes armrests 316 that extend from the backrest 310. FIG. 3A illustrates how the motor and control systems disposed at least partially in the horizontal support 306 can lower right leaf 308A, while maintaining a horizontal position to left leaf 308B. In FIG. 3B, both right and leaf 308A, 308B are lowered to be vertically aligned with the vertical support 304. These different configuration positions of the seat portion, which is comprised of right and left leaf 308A, 308B, can be controlled using at least control interface 312. Other mechanisms of controlling the positions, timing, and other adjustments of the seat portion will be described below.

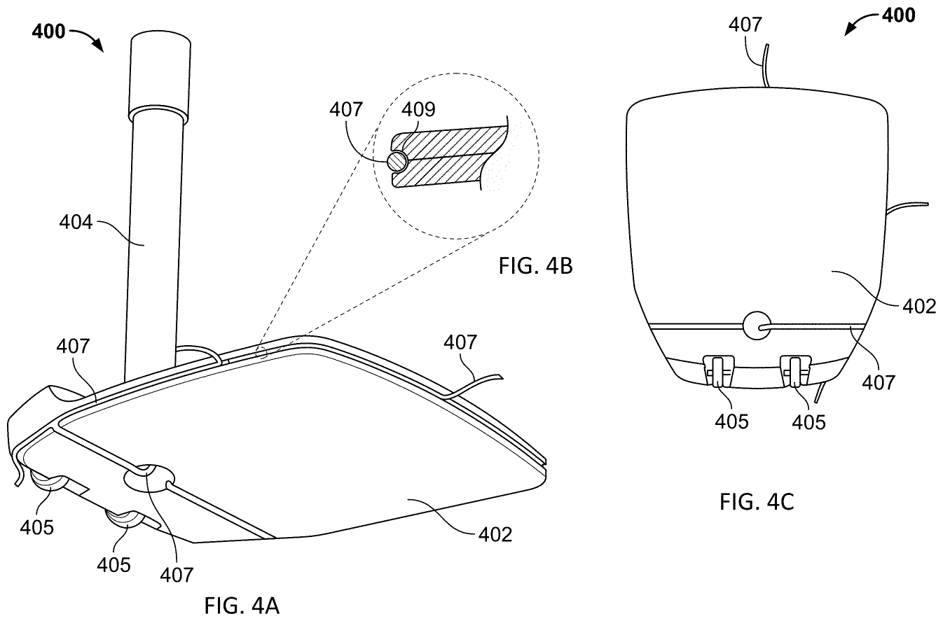

FIGS. 4A-C illustrate another embodiment of an intelligent automated chair 400, that illustrates routing of power through the base 402 and the vertical support 404. As shown, various channels 409 can be formed on the underside of the base 402 as well as along the edges of base 402 to guide an electrical cord 407 that can provide power to the intelligent automated chair 400. Also shown are wheels 405 that are attached near the back portion of the base 402 that can be used when moving the intelligent automated chair 400. It should be understood that the various features of embodiments 100-400 can be integrated across each other as well other embodiments that will be described later.

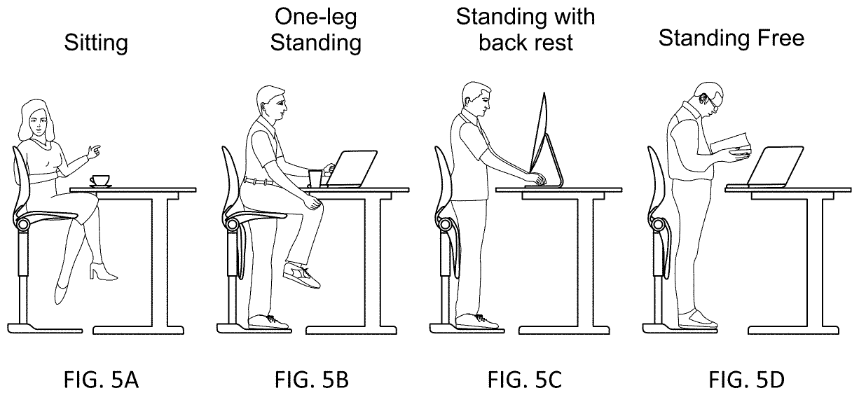

FIGS. 5A-D illustrate a change in position of an intelligent automated chair and the various configurations or positions a user could position themselves about the intelligent automated chair. For example, as shown in FIG. 5A, a user could be fully seated on the intelligent automated chair. In this fully sitting position both the right and left leaves are fixed in a horizontal position. After a period of time, the user can transition to a position shown in FIG. 5B where either the right or left leaf is lowered. In this position the user is standing on one leg (either the right or left leg) while resting the other leg or approximately half of the buttocks on the leaf of the seat that is in the horizontal position. The user can alternate standing on one leg while partially sitting from right side to left side. FIG. 5C illustrates another position the user can utilize the intelligent automated chair where the user is in a standing position, but still leaning their backside on the vertical portion of the intelligent automated chair. In FIG. 5D, the user is standing free of the chair on the base area, and not leaning on the intelligent automated chair at all. By switching between each of these positions the user can benefit from some of the health benefits noted above.

FIGS. 5A-D illustrate also that the intelligent automated chair can be used with a desk or table or type of workstation, which should be readily understood from reading this description. A workstation generally includes a chair, desk, computing device, monitor and other various office supplies.

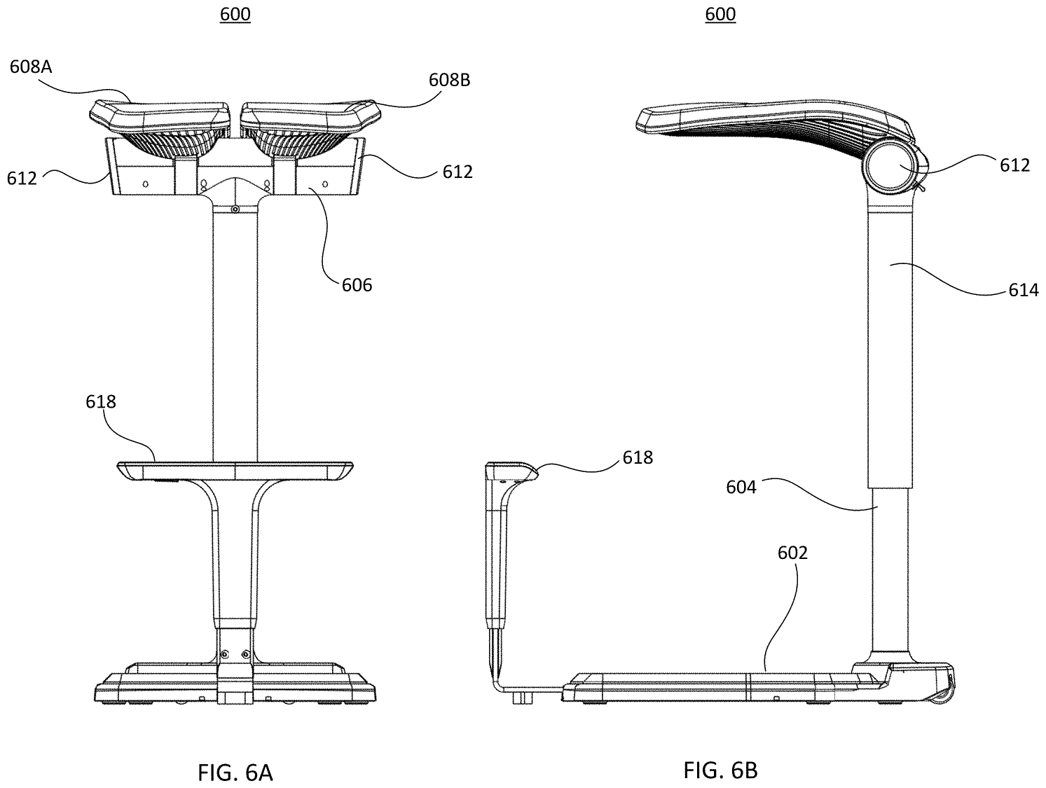

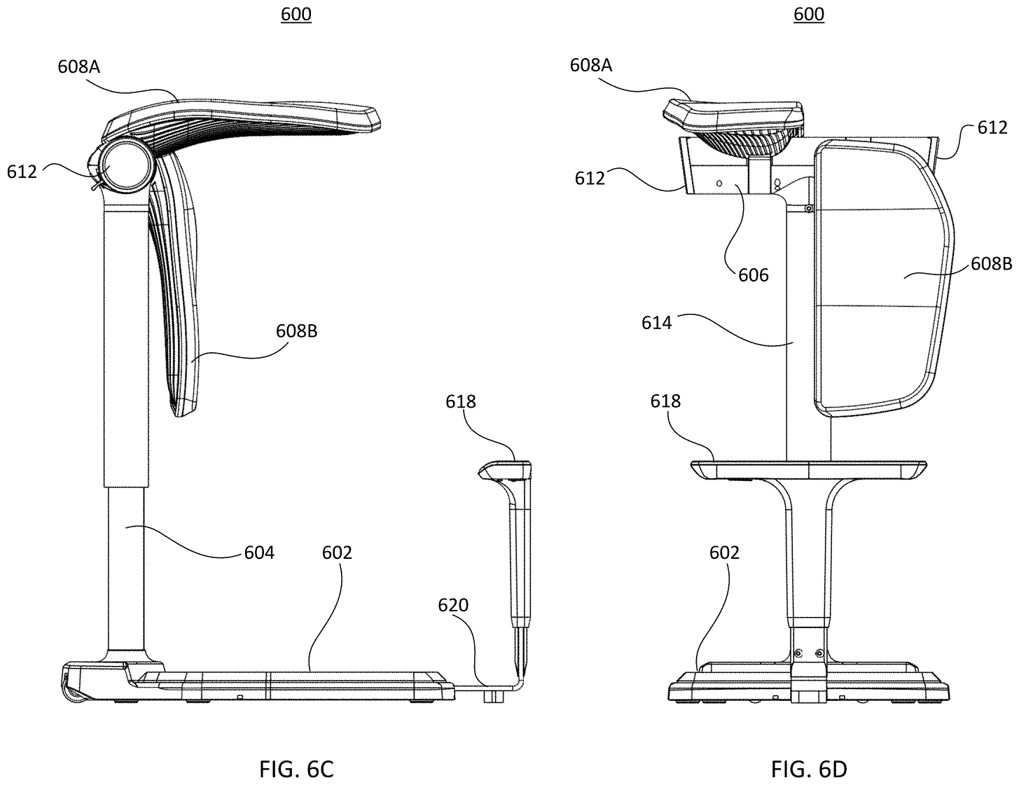

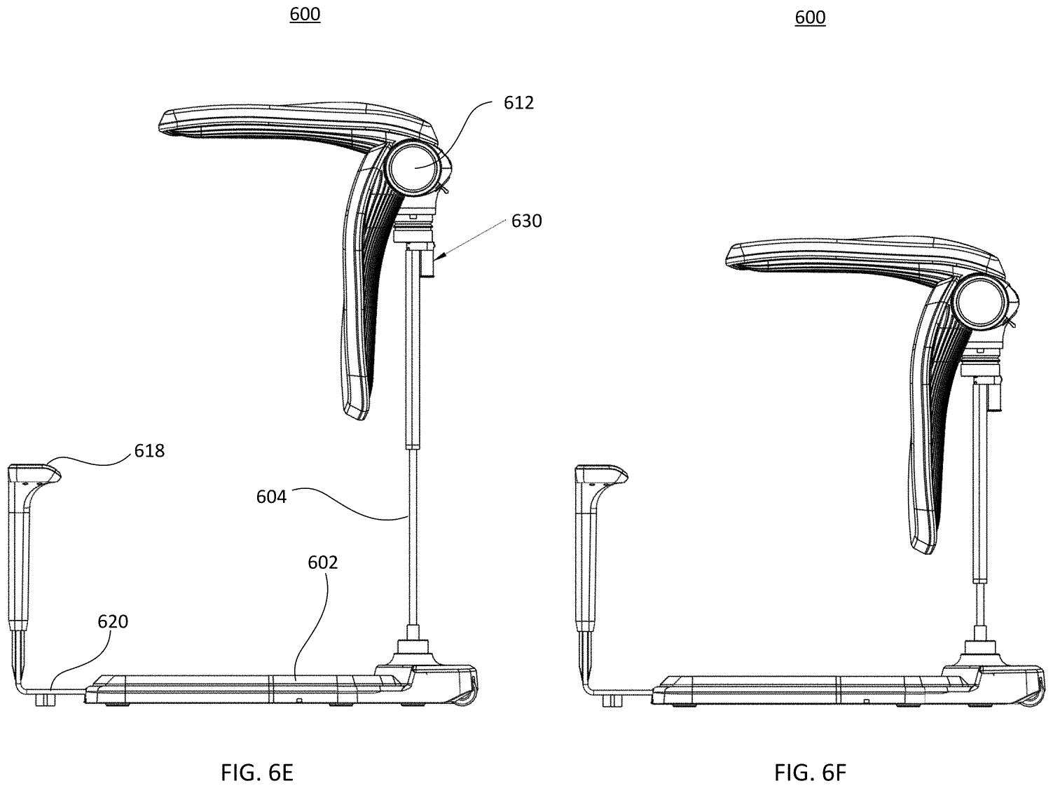

FIGS. 6A-F illustrate various views of an intelligent automated chair 600 without a backrest. Similar to the embodiments described above, chair 600 includes a base 602, connected to a vertical support 604, which is connected to the horizontal support 606, which includes the automated control assembly therein, which is comprised in part of motors and a control system. The horizontal support 606 is mechanically connected to the right leaf 608A and left leaf 608B, which form the seat and are raised and lowered by the automated control assembly. As noted, this version does not include a backrest or armrests. However, this version includes a foot rest 618 that includes a foot rest adjustment mechanism 620. The foot rest 618 feature can be incorporated into any of the above embodiments. The foot rest 618 is designed to allow a user to rest their feet on when in the full sitting position. The foot rest adjustment mechanism 620 can be a manually adjustable mechanism, which is configured to extend the foot rest further away from the seat or can adjust the height of the foot rest. It can also be an automated system, such as the vertical height mechanism 614 disposed internally in the vertical support 604 and driven by the motor and control system.

FIG. 6A illustrates a front view of intelligent automated chair 600 in a configuration where both the right and left leaf components are both upright. A side view of this configuration is shown in FIG. 6B. FIGS. 6C-D illustrate 600 in a configuration where the left leaf 608B is lowered, while right leaf 608A is maintained in a horizontal position. This allows the user to be in the on-leg standing position, where the left leg is standing and the right leg is sitting or resting on the right leaf. FIGS. 6E-F illustrate a configuration where the vertical support 604 has an integrated electric linear motor 630 integrated therein. The electric linear motor 630 can allow the height of the intelligent automated chair 600 to be automatically raised or lowered. This automatic raising can be done by user input, such as user input to one of the control interfaces 612, or user input into a wireless connected device running an app.

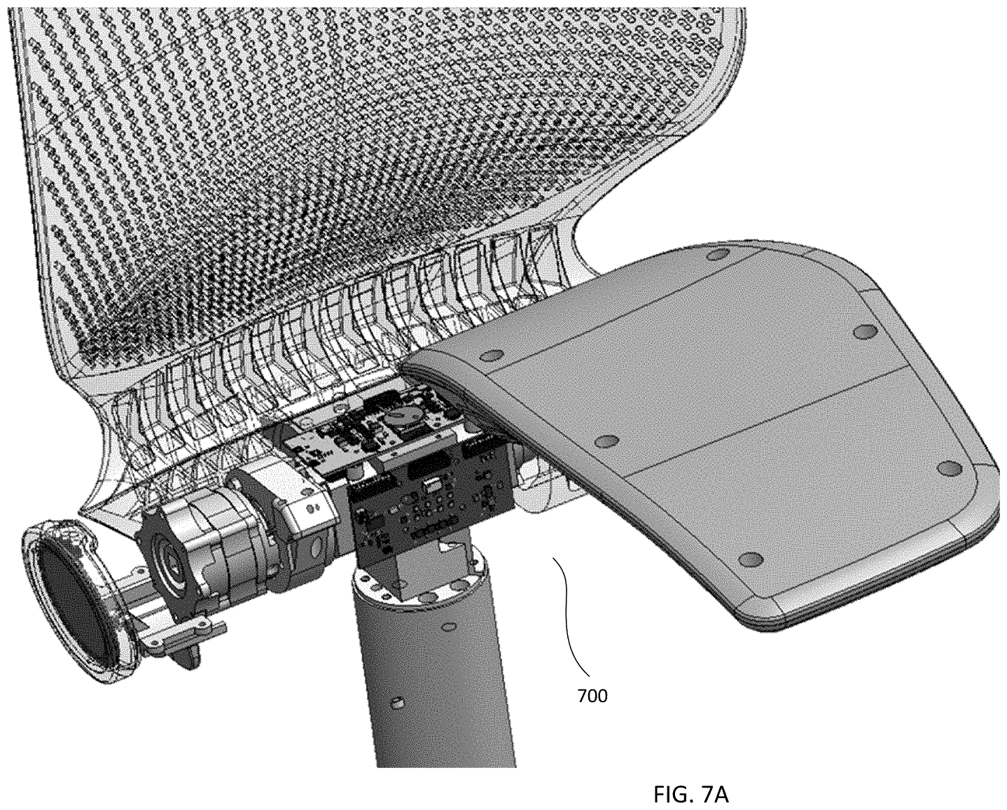

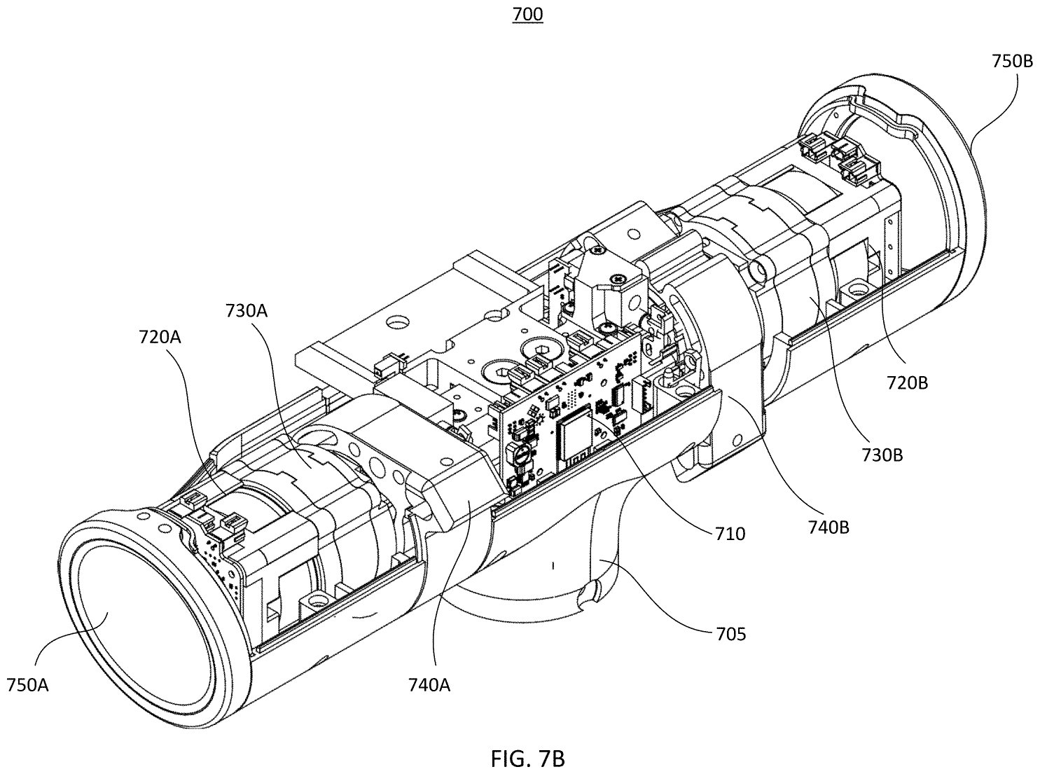

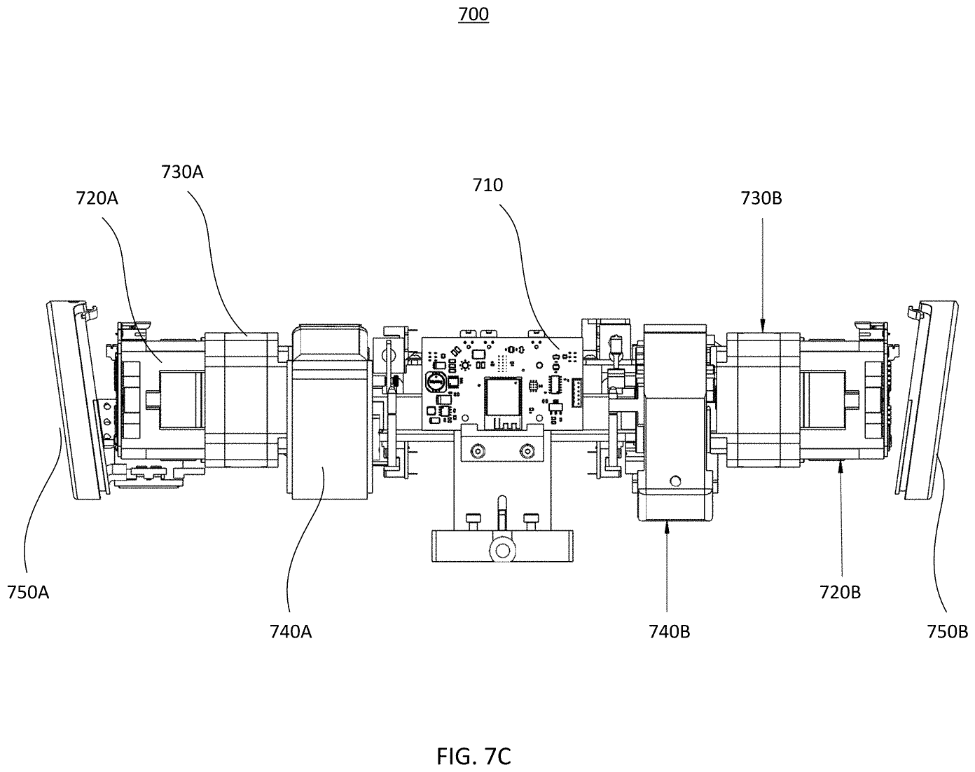

FIGS. 7A-C illustrate various views of the automated control assembly 700 used in the intelligent automated chairs. FIG. 7A illustrates a partially cut-away view of the automated control assembly 700 that is housed and integrated with the horizontal supports noted above. The automated control assembly 700 as shown in FIGS. 7B-C have a control system 710 and two motors 720A, 720B. The motors 720A, 720B can be brushless DC motors. These can be connected to and operate gearboxes 730A, 730B, which in turn interface with output controllers 740A, 740B that connect to the right and left leaves of the seat. An interface 750A, 750B are shown on the opposite ends of the assembly 700 and can utilized as an input interface for the assembly 700. A vertical support interface 705 is shown and configured to attach to the vertical supports previously described and shown.

The raising and lowering of the seat halves (right leaf/left leaf) are controlled and powered using the control system 710. The control system 710 can include one or more processors, memory, logic, power supply, sensors, wireless communication means, such as antennae configured to transmit and receive Bluetooth and WIFI protocols and signals. The control system 710 can further receive instructions on how to operate the controls leading to the changing of chair configurations, as shown in FIGS. 5A-D. For example, a set of operating mode instructions can be received wirelessly by the control system 710 and stored in an executable format in memory or logic to operate according to those operating mode instructions. As will be discussed in more detail below, the control system can also receive real-time feedback from the interfaces 750A and 750B, from one of the sensors, or interference when a change of position occurs to alter at least temporarily the current operating mode. This real-time feedback and input can also be used to update the current operating mode. The updating the pattern and operating mode calculation can either be performed in the control system, sent to a mobile computing device (or even the cloud) to be updated and then overriding or updating the instructions associated with the original operating mode. The control system 710 can also store usage information for later offloading and analysis in the cloud. This usage data can be part of a historical information database that include individual and/or group historical information, which is used to train and update recommended operating modes to users. It should be noted the operating modes determine the frequency of position of changes, the pattern of the position changes, the duration (e.g., 30 seconds standing, 1 minute fully sitting, 45 seconds right leg standing, 30 seconds left leg standing) of each position, the type or style of change notifications, default position when interrupted, and so forth.

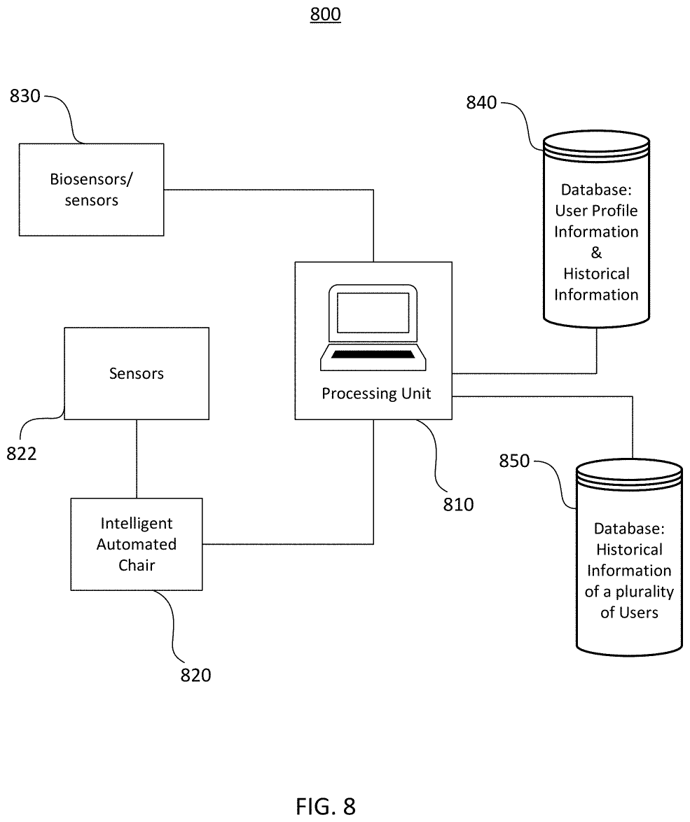

FIG. 8 illustrates a schematic of a system 800 of an intelligent automated chair using various forms of input to determine the operation of the intelligent automated chair. As shown, a computer having a processing unit 810 can receive external biosensor and other external sensor input 830, historical usage and profile information associated with the user from database 840, historical usage and profile information associated with a plurality of users from database 850, which can be used to generate an operating model for the intelligent automated chair 820. As noted above the chair 820 can further receive direct input from sensors 822. These sensors can either be integrated with the chair 820 or the sensor input information can be received directly, such as via wireless communication means.

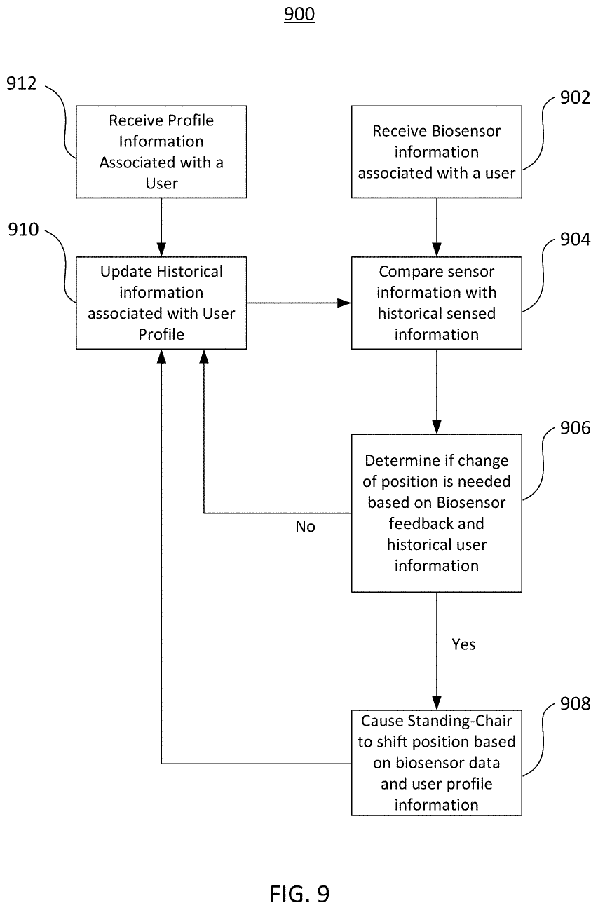

FIG. 9 illustrates a workflow 900 indicative of at least one mode of operation determination. As shown, biosensor feedback 902 can be received when a user is using the intelligent automated chair, which can be compared 904 with historical biosensor information and user profile information associated with the user, an analysis 906 can be performed to determine whether or not the chair should change positions, patterns, or frequency of position changes. If the determination is positive then it is implemented in step 908 and that information is updated and stored 910 as part of the user's historical information, which incorporates user profile information 912 received by the user. It should be noted that this workflow can be performed while a user is using the intelligent automated chair, or performed at another, which then updates the operating model to be implemented the next time the user uses the intelligent automated chair.

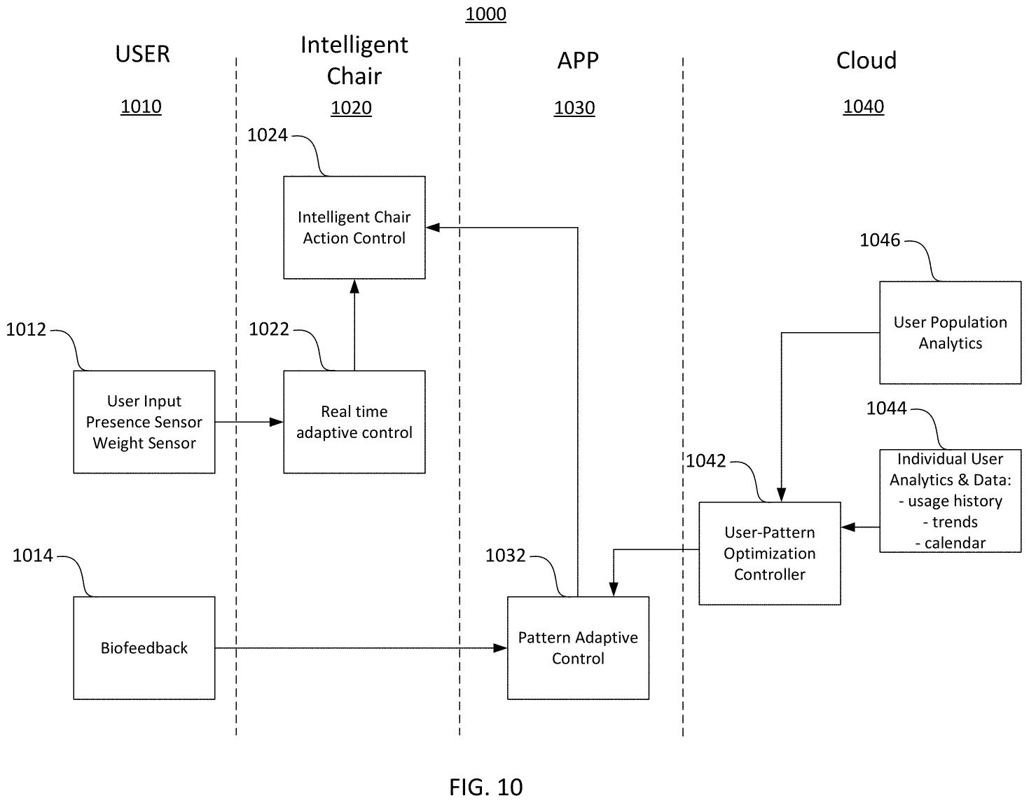

FIG. 10 illustrates a schematic where various input and decisions can occur in the intelligent automated chair system 1000. In one embodiment the intelligent automated chair system 1000 includes an intelligent automated chair, means to receive biosensor and other sensor information, the cloud to use stored information to generate models and recommendations and an app to interface and implement those models. In the User column 1010 the boxes 1012 and 1014 are placed indicating this is information received about the user or directly from the user, which includes receiving: user input, presence sensor information, weight information in box 1012 and biosensor information in box 1014. The information of box 1012 can be received and utilized by a real-time adaptive controller 1022 that is associated with or integrated as part of the intelligent automated chair shown in the intelligent chair column 1020. This real-time adaptive controller 1022 can process the information in real-time and then communicate with the intelligent chair action control 1024 to implement any changes in how the intelligent chair is operating. In the App column 1030, which is used to illustrate a software application, a pattern adaptive control module 1032 is provided to update the intelligent chair action control 1024 based on analysis of information performed by various modules in the cloud column 1040. As shown, a user population analytics module 1046 can receive and send such information to user pattern optimization controller 1042, which can also receive information from the user analytics module 1044. The user pattern optimization controller 1042 can generate an optimized pattern for an operating mode for a particular user. It should be noted that the user analytics module 1044 can include usage history of a user, trends of user, and even calendaring information associated with a user. For example, when the user is in meetings, the historical information can identify or include information with how the user interacts with the intelligent automated chair, which could be different when the user, who for instance could be a programmer, is writing code. Thus, the calendaring information which can compare previous interactions with the user during specific type of events and can also use that same information to generate an operating model that adapts to those future events listed on the user's calendar. All of this is fed into a user population analytics module 1046, which in turn is sent to user pattern optimization controller 1042 and on to the pattern adaptive control 1032 which can additionally incorporate biosensor information on top of the layer of pattern optimized information received from the cloud column.

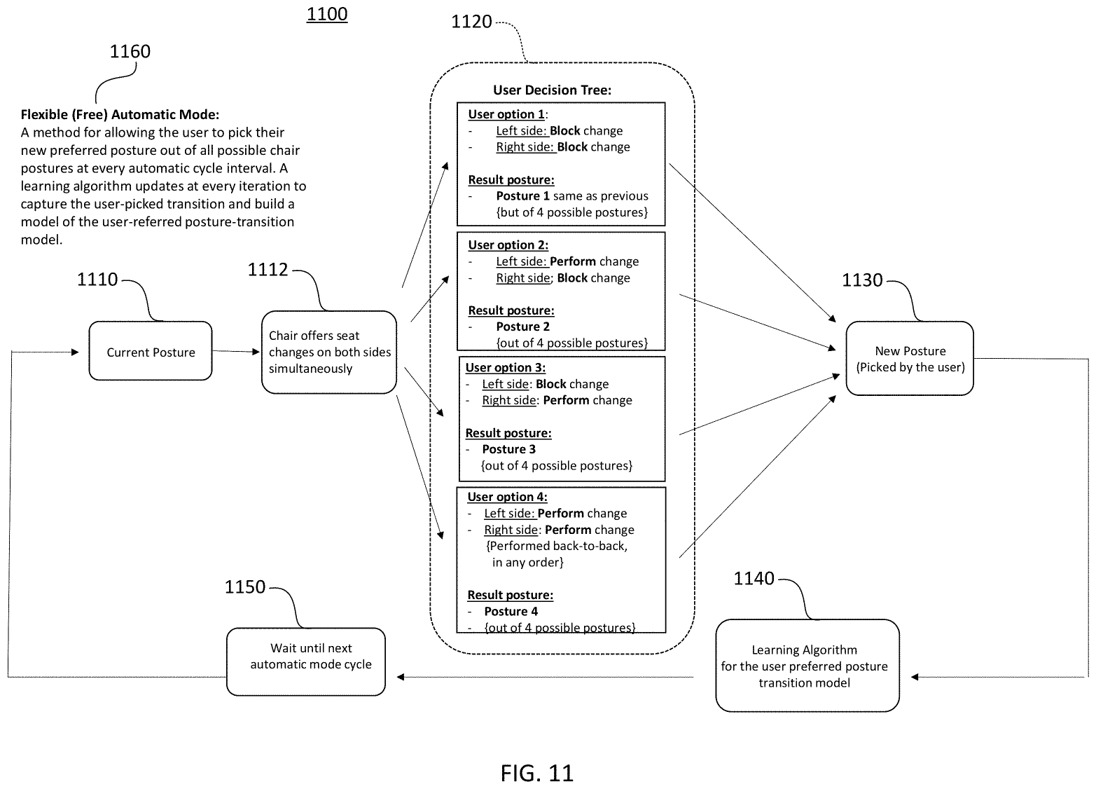

FIG. 11 illustrates one mode 1100 of operating the intelligent automated chair. This operating mode 1100 can be a flexible automatic mode 1160, which includes the ability to run an automated operating model on the intelligent automated chair that has the ability to be interrupted and take real-time feedback that can be used to update the current automated operating model. As shown in the flowchart, the intelligent automated chair has a current position of 1110. The user has the ability to alter or change the current position as the intelligent automated chair is configured to receive user input 1112 in the middle of an automated cycle. The decisions the user can make are shown in the user decision tree of 1120. For example, if the current position is a sitting position, the user can decide under option 1, to block change on both the right and left side, where the intelligent chair then maintains the current position determining the posture of the user. Under user option 2, the user can elect to change the left side or left leaf and block any change associated with the right side. Again, if the initial position is sitting, then the position changing to a one-leg standing position, where the left leg is standing and the right leg is resting on the right leaf. Under user option 3 the user can do the opposite, so then the left leaf remains in the same position and the right leaf alters. If the initial position were right leg standing, then with this change the user would transition to fully sitting. Under user option 4 the user has the ability to change the current position of both the left leaf and right leaf. Thus, if the previous position were sitting, then this transitions the user to a standing mode. Once the input is received with regards to each leaf the new position is implemented in step 1130. This information of the change can then be transmitted to a learning algorithm 1140 that determines an updated way of operating that can be incorporated into the operating model, which can be implemented in step 1150 until the next automatic mode cycle happens where the circular flowchart is completed to the current position of the chair.

It should be understood that the user input can be performed in a variety of manners. One example, includes the user interfacing with control interface (112, 212, 312, 612) to determine the next position of the intelligent automated chair. Another example of the user providing input includes using more natural inputs that take advantage of various sensors implemented into the intelligent automated chair. Another form of user input, can include the user giving a voice command to a mobile computing device, which is communicated to the intelligent automated chair. Another includes selecting a new position on a control interface running on an app on a mobile computing device.

With regards to natural inputs, these can take advantage of natural user interactions. For example, if the user wants to transition from a fully sitting to a one-legged standing position the user can place their hand under the right or left leaf and lift up on the particular leaf. This input can be detected by a load detection system associated with each side. When the load sensor determines that there is an upward load it can then release the appropriate side and drop the leaf down, so the user transitions to a one-legged standing position. Another natural input can include the user reaching with the back of the foot or ankle to pull on the leaf (that is in a down or vertical position), which again can be detected or sensed by the load detection system and then cause the particular leaf to begin raising to a horizontal position. An example of a natural user input intended to block a position can include not shifting or releasing load from the right leaf, left leaf, or both sides. This can additionally include slightly pushing down on one or both sides to block the change. When the right or left leaf is trying to raise up and the user wants to keep standing, the user can push back slightly using their leg and such change in load can be detected to keep or return the leaf to the vertical position.

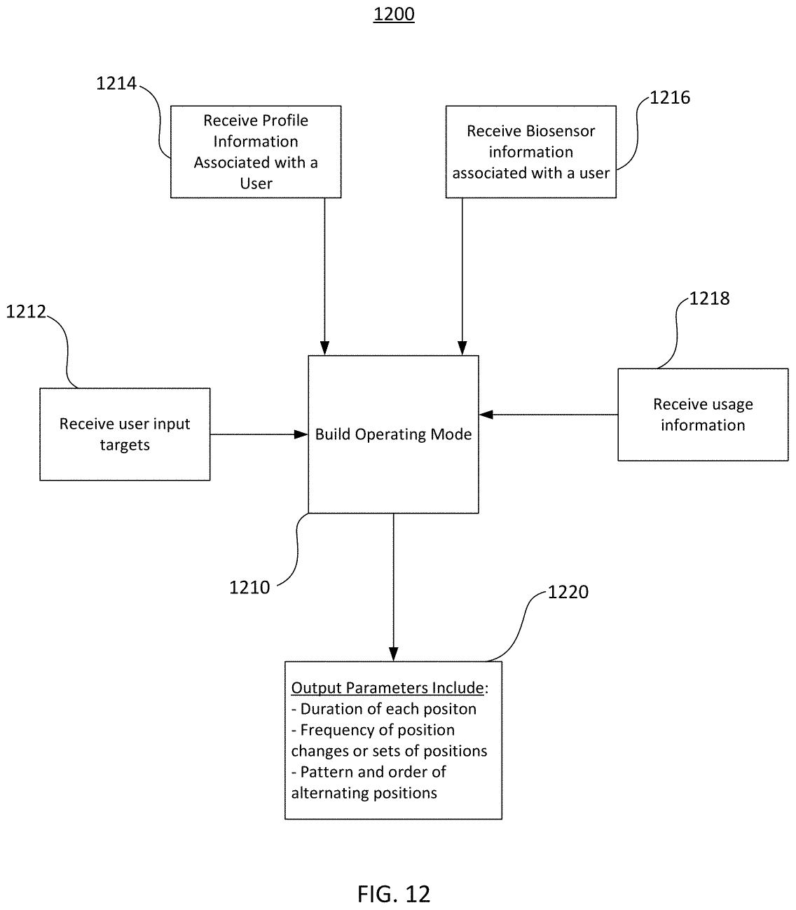

FIG. 12 illustrates a workflow 1200 for creating an operating mode and the parameters associated with the operating mode. Here the method includes receiving user input targets in step 1212, receiving profile information associated with a user in step 1214, receiving biosensor information associated with the user in step 1216, and receiving usage information in step 1218. This information can then be used to build an operating mode 1210 for the intelligent automated chair to operate from. The output parameters 1220 of this built operating mode can include the 1) the duration of each position, 2) the frequency of position changes and 3) pattern of rotating positions. It can also include certain targets and thresholds (min or max) to achieve. For example, the user could input a target to stand 10 minutes longer today than yesterday. There could be a target to try and keep a heart rate up a certain percentage, which can translate into changing positions more frequently or utilizing more standing positions than sitting positions. These types of user input targets are exemplary and not limited, as one of ordinary skill in this art would recognize a bevy of other types of targets or goals a user could input. These targets or goals can be daily, weekly, and so forth.

User profile information can include a variety of information such as height, weight, gender, preferences, type of job, activity level, and so forth. This information can be updated and upon updating can be used to update their user operating mode. It should also be understood that a single user can create their own operating mode by manually selecting the patterns, durations and so forth. A user could have any number of operating mode profiles that they create and can be associated with their user profile. The user can select any of their stored operating mode profiles to run using the app.

A method of training the intelligent automated chair system includes, operating the intelligent automated chair in a training mode. The training mode is configured to monitor the pattern usage of the user and how they interact with the chair. This training mode usage information can then be used to recommend operating mode for the user based on the training mode. The recommended operating mode generated from the training mode usage information can further receive profile and user input target information to generate the recommended operating mode. The system can also receive other historical information associated with other users and particularly usage information of others where the user is an early or first-time user of the system.

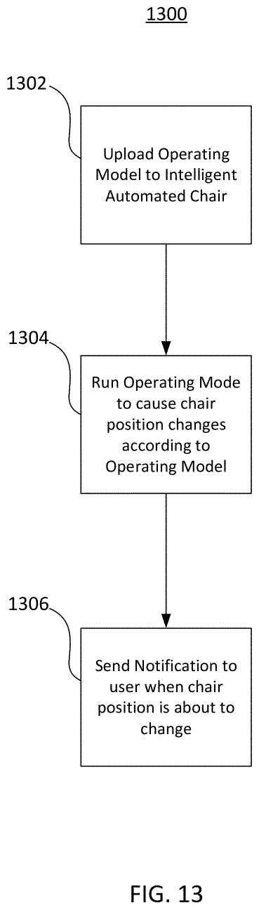

FIG. 13 illustrates a flowchart 1300 where the user receives a notification prior to the intelligent automated chair changing positions. The intelligent automated chair is configured to receive or have operating model uploaded to it in step 1302. In step 1304, the intelligent automated chair begins running the operating model. In step 1306, the user receives a notification prior to and when the chair is about to change positions. This notification can come in a variety of formats and is determined by the user. The notifications can include, the right leaf or left leaf vibrating, an audible sound from the intelligent automated chair or smartphone, a light notification from the intelligent automated chair, smartphone or other connected device, or a physical contact by the intelligent automated chair. This physical contact can include the right or left leaf beginning to raise up and engaging the user on the back of the leg indicating that the right or left leaf is about to raise to a horizontal position.

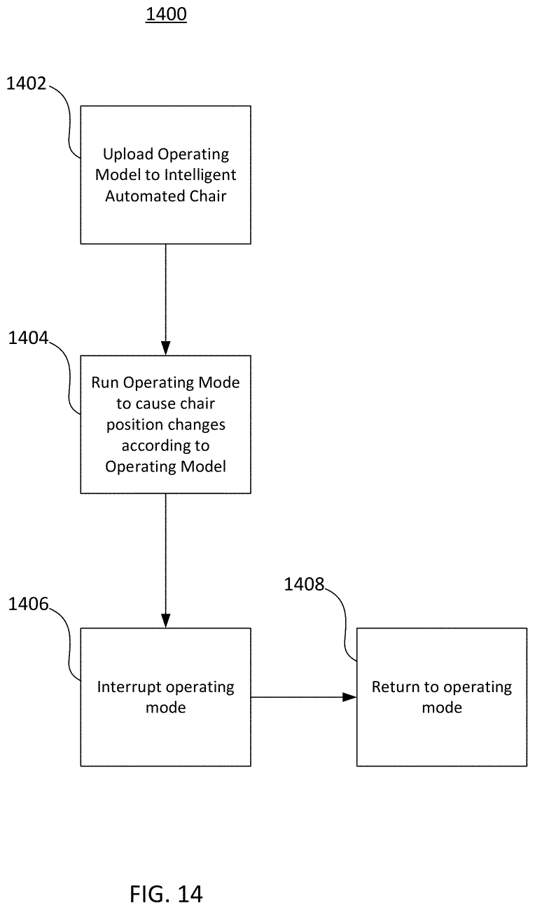

FIG. 14 is a flowchart 1400 that illustrates the interruptible nature of the intelligent automated chair. The intelligent automated chair is configured to receive or have operating model uploaded to it in step 1402. In step 1404, the intelligent automated chair begins running the operating model. Once the intelligent automated chair begins cycling through the position changes it can be interrupted in step 1406 for a period of time. This interruption can be the result of the user manually blocking a change, it can be an automated result of sensor determining the user is blocking the change through a sensed means, or it can be interrupted as a result of a pre-defined setting that blocks the change whenever a particular event or sensed event occurs. For example, the operating mode could be interrupted when an overhead announcement occurs, a phone call is received, another user is detected nearby, or an event such as a team meeting occurs on the user's calendar. After the interruption, the operating mode can resume its normal operating mode in step 1408.

As alluded to above, each time a user shifts positions in the intelligent automated chair, the motion can be sufficient to shift the pressure on certain parts of the user's body, such as the spine or lower back, to other parts of the user's body that allow for increased blood flow to different muscles and portions of the spine. This can help to increase blood flow to those parts of the body and keep them from becoming overstrained. The motion can also cause the heart to increase the number of beats per minute. This shifting is not akin to running on a treadmill, using other cardio equipment or strengthening equipment. A user might be able to consume information while running on a treadmill for example, but creating information becomes very difficult. The focus of cardio and strength training equipment is to reach target heart rates and optimize caloric burning. That is different from the present system and methods where the objective is to optimize health benefits while maintaining, if not increasing, focus and creativity needed to perform various desk type jobs at a user's workstation as noted above.

Another one of the advantages of the system and methods described herein includes the ability for a user to alter or interrupt at their control. An intelligent automated chair can run an optimized operating model based on the various inputs described, but the user can still have control over the automated profile at any given moment and adjust or take control accordingly. Thus, allowing for the most amount of freedom or flexibility when using the intelligent automated chair.

The bio-sensed data can be received from a variety of sources including: smartwatches, smartphones, pressure sensors in the chair, IR sensors about the workstation, and other wearable devices that can track bio-sensed information.

In the various embodiments, local sensors provided about the equipment can include pressure sensors, accelerometers, flow sensors, strain sensors, humidity sensors, temperature, sound, and optical sensors.

The automated control assembly can provide instructions and incorporate with a haptic feedback driver module, which controls the haptic feedback controls of the intelligent automated chair. These haptic controls and sensors can be incorporated into various parts of the intelligent automated chair including, but not limited to: the back rest, the right and left leaves, the base, the foot pedestal (if any), a user input control module, the crossbar and support bar, and so forth. Some of these will include servo-motors or electric motors, others will be sensors, and some will include power electronics.

Some of the haptic controls and sensors can determine how much of a user's weight is resting on the standing leg as compared to the resting leg. If a user is not within a designated range (either determined by the user or recommended by the system) a haptic (or other style) of notification can occur indicating to the user to shift their weight. For example, if almost 80% of the weight of the user is placed on the standing leg, and the determined range is to not exceed 60% percent for more than a specified duration, like more than 5 seconds, then the system can create a notification to the user to shift more weight to the resting leg.

This transferring of weight and using one leg more than another can be a part of the usage information that is displayed on the app under the user's profile or account. This can be yet another type of user input target manually selected by the user or automatically recommended by the system to train the user to balance more or strengthen one side of their body over another. For example, if a user heavily favors one leg over another, this could be indicative that their back is out of alignment and needs adjusting and strengthening. With this information the user can select an operating model or put a target input to have an operating mode updated to help facilitate this change, which might include standing more often on the weaker leg as opposed to the stronger leg.

Another aspect of the present invention is that the sensors can determine when the user is approaching and lower one or more of the chair leaves depending on the side the user is entering to engage with their workstation. Multiple user profiles can be associated with a single automated chair. Bluetooth enabled, as well as WIFI enabled watches, smartphones and other devices can communicate with the chair to modify other settings based on the user approaching to use the automated chair such as preferred operating mode profile.

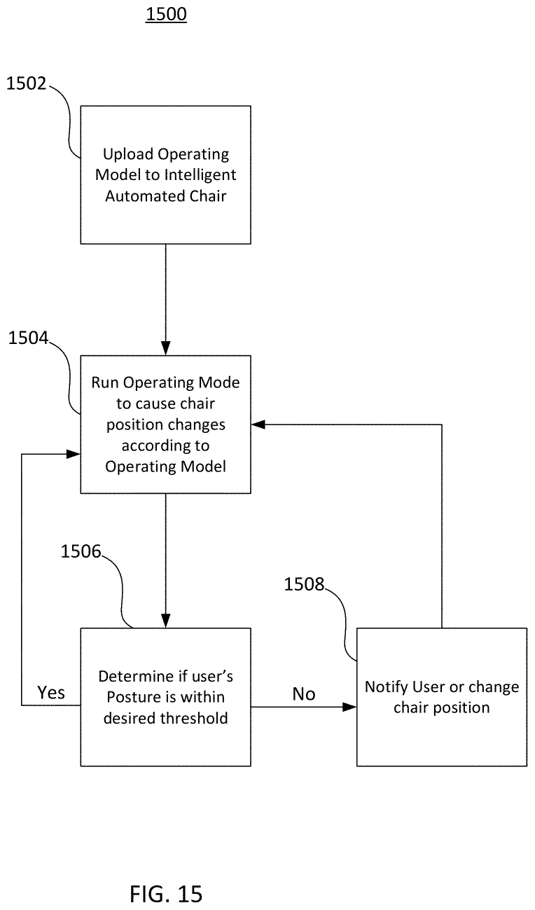

FIG. 15 illustrates a flowchart 1500 of notifying the user or interrupting the operating mode based on sensed posture information. The intelligent automated chair is configured to receive or have operating model uploaded to it in step 1502. In step 1504, the intelligent automated chair begins running the operating model in an operating mode. In step 1506 the system using various sensors, that can include pressure and weight sensors, to determine if the user is slouching or has an appropriate posture. If it is determined that user does not, then in step 1508 the user can be notified to change their posture through the various notification mechanisms described herein or cause the system to create an immediate chair position change.

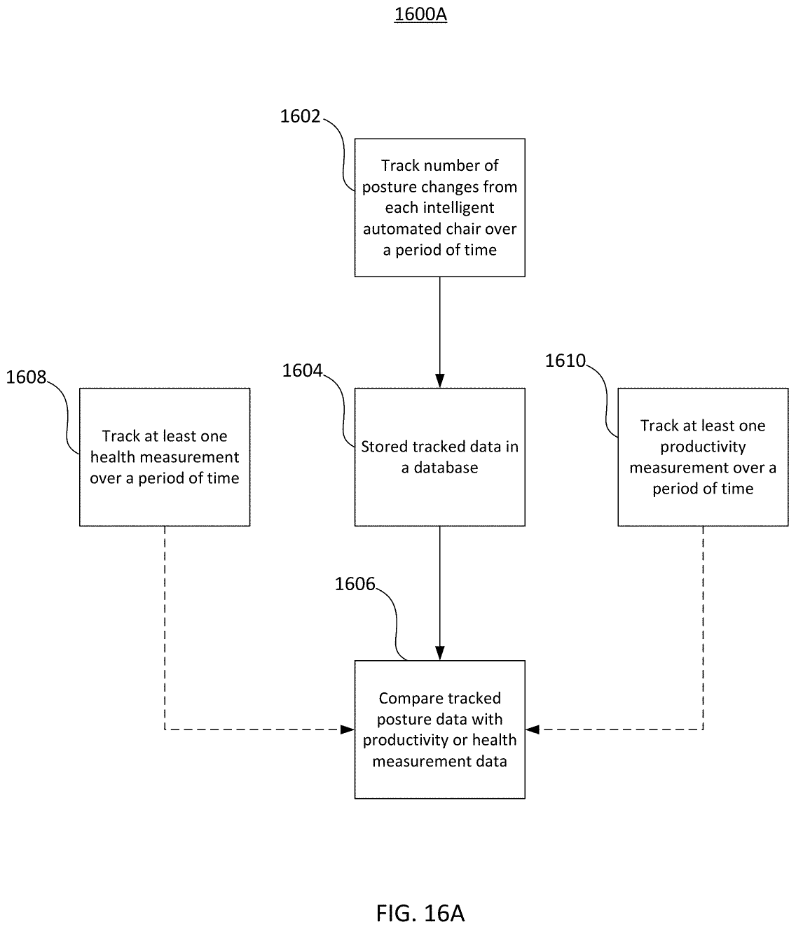

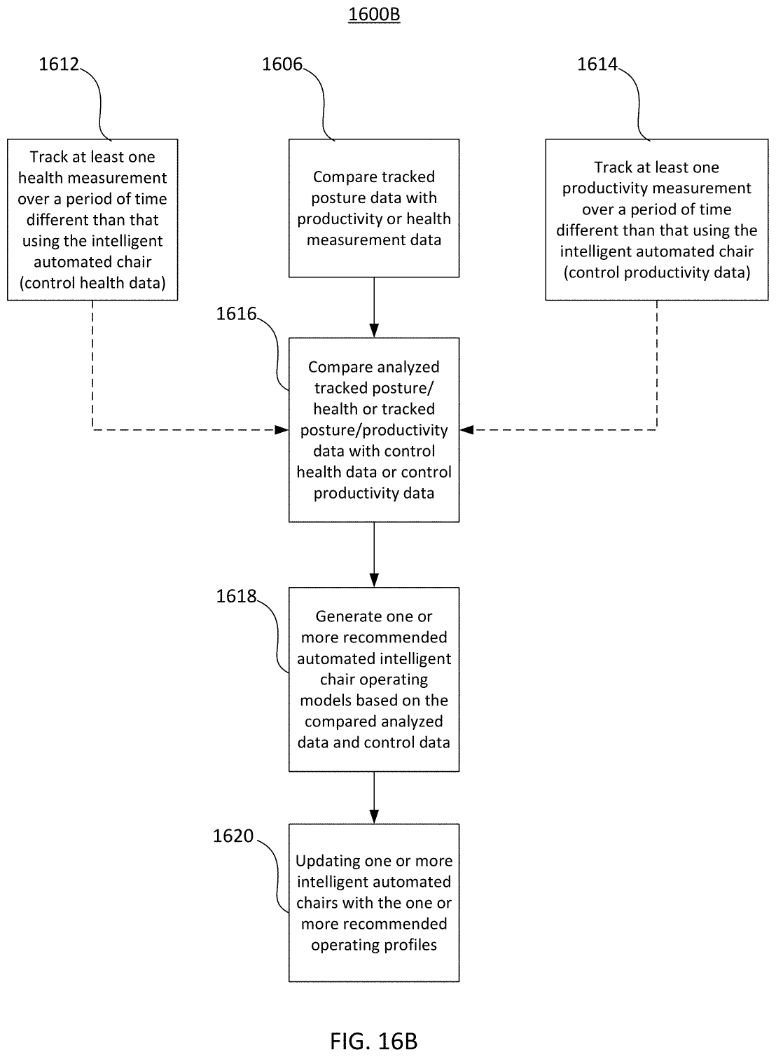

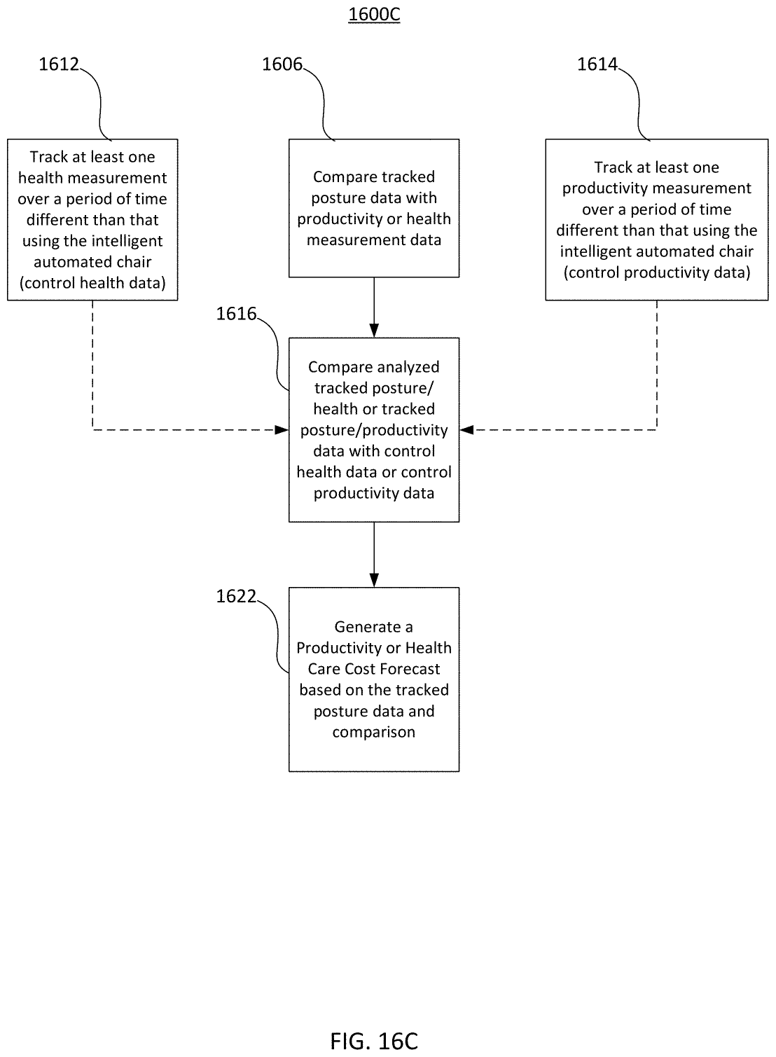

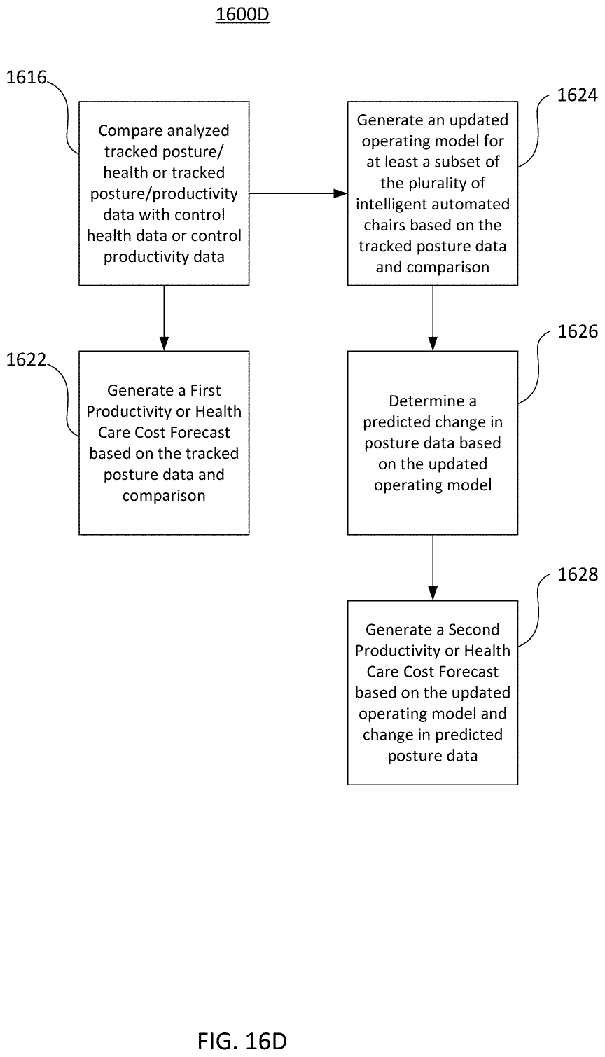

FIGS. 16A-B illustrate various workflows for determining the number of posture changes for a group and providing one or more recommended posture change operating models. Regarding the workflow 1600A, shown in FIG. 16A, the number of posture changes from each intelligent automated chair, such as one of the many embodiments described above, is tracked during step 1602. Each of these tracked postured changes can then be stored in a database during step 1604. Once tracked posture changed data is tracked over a period of time, it can be compared during step 1606 with the health and/or productivity measurement data tracked for the same period time during steps 1608, 1610.

Examples of the health measurement data can include number of sick days taken by the group, group costs spent on medical care, types of medical care needed for the group, number of health claims the group had, number of work-related injuries the group had, type of work-related injuries the group had, costs the group spent on prescriptions, heart rate information, blood test information (which can measure various things such as cholesterol, iron, sugar, insulin, metals, and so forth), body weight (individual and group, including average weight over a period of time), amount of coffee consumed by the group, and amount of water consumed by the group. This list is meant to be exemplary, as health measurement data can include any set of measured data related to health.

Examples of the productivity measurement data can include sales for the group, customer support metrics, periodic reviews, periodic ratings, project completion timelines, work quality, attrition rate, keystroke measurements, typing accuracy, and production output. Again, this list is meant to be exemplary, as productivity measurement data can include any set of measured data related to productivity of a business or organization.

Regarding the workflow 1600B, shown in FIG. 16B, control health data sets can be tracked during step 1612 that is for a period of time separate from that overlapping the tracked posture change data period of time. Similarly, control productivity data sets can be tracked during step 1614 that is also for a period of time separate from that overlapping the tracked posture change data period of time. These control data sets can then be compared against their counterpart health or productivity data sets that coincide with the tracked posture change period of time during step 1616. In some instances, the tracked posture change data could be during an initial implementation or usage phase of the intelligent automated chairs. Once these control sets of data are compared with the posture tracked data during step 1616 the system can utilize a learning algorithm as well as historical data from other sources to generate one or more recommended automated intelligent chair operating models during step 1618. These operating models 1620 can then be uploaded onto one or more of the automated intelligent chairs being used by the group. This cycle can then be repeated. For example, once the updated profiles have been deployed the system can then track the posture changes during a new period of time using the updated profiles, and the health and productivity measurement data can again be tracked and recorded during this new period of time. The system can then compare this against the control set, the original implementation as well as the updated chair operating model period to again determine whether a new operating model is to be recommended and once again implemented.

It should be noted that one of the advantages of utilizing the systems and methods described herein, is they enable a user or company to customize an operation model of the intelligent automated chairs described herein to better help address absenteeism, which is the abnormal or excessive missing of work days, some of which can be related to the overall health of an individual, which the intelligent automated chairs are designed to improve upon, as well as presenteeism, which is the focus and engagement level of a given employee that can often be measured using various productivity measurements. These are in addition to addressing reducing overall health costs, and increasing productivity for a company, group of users, or even an individual.

It should be readily understood that certain metrics can be focused on, which are then used in a weighted manner as part of a formula to optimize an operation model. As more metrics are determined, some may have differing optimal ranges. For example, for the best blood flow and reducing the most amount of back pain, change posture positions every 2 minutes, might be ideal. However, the data may suggest that optimized productivity occurs in a range between switching every 4-8 minutes, with 8 minutes being preferred. These are by way of example and are not the results of actual data. Thus, by balancing the metrics, and weighting them the result creates an operation model that causes posture changes every 3 minutes.

The operation model can be altered in more ways than duration of time, as specific posture positions might also have an impact on productivity or health and might be individually preferred across subgroups of the overall group. Though a lot of the focus has been on quantity of posture changes, the type and duration of each can also be utilized and tracked, so as to be compared with the other tracked metrics described above. Each of these can then be part of the optimization algorithm configured to determine an optimized operation model based on the desired outputs.

FIGS. 16C-D illustrate various workflows for determining the number of posture changes for a group and providing one or more health or productivity forecasts. In particular workflow 1600C uses similar steps of 1606, 1612, 1614 and 1616 as workflow 1600B; however, here this tracked and compared information can be used to generate a productivity or health cost forecast as shown in step 1622. The forecasts can be based on controlled data, without the intelligent chair, compared to data resulting in using the chair using an initial operation model.

FIG. 16D illustrates workflow 1600D that starts with step 1616, and from here can also use this information to generate a first forecast 1622 as in workflow 1600C, but this compared information from step 1616 can also be used to improve or generate an updated operation model for at least a subset of the overall group in step 1624. With the new operating model, a step can then be performed to determine the change in posture changes in step 1626. With the predicted change generated from step 1626 that information can be used to predict a second health or productivity health model based in part on the posture change information in step 1628. These forecast models can further be compared in an additional step (not shown). The forecasting data can be useful to help negotiate various insurance rates for companies, as the tracked data and models can generate quantifiable and correlated information that has a direct impact on costs. These forecast models can likewise be used to induce certain behaviors in various industries. Similarly, the forecasting models related to productivity can be utilized in various to help secure better interest rates for loans, determine growth for the company, and used in a variety of business-related manners some of which can increase the overall value of the business.

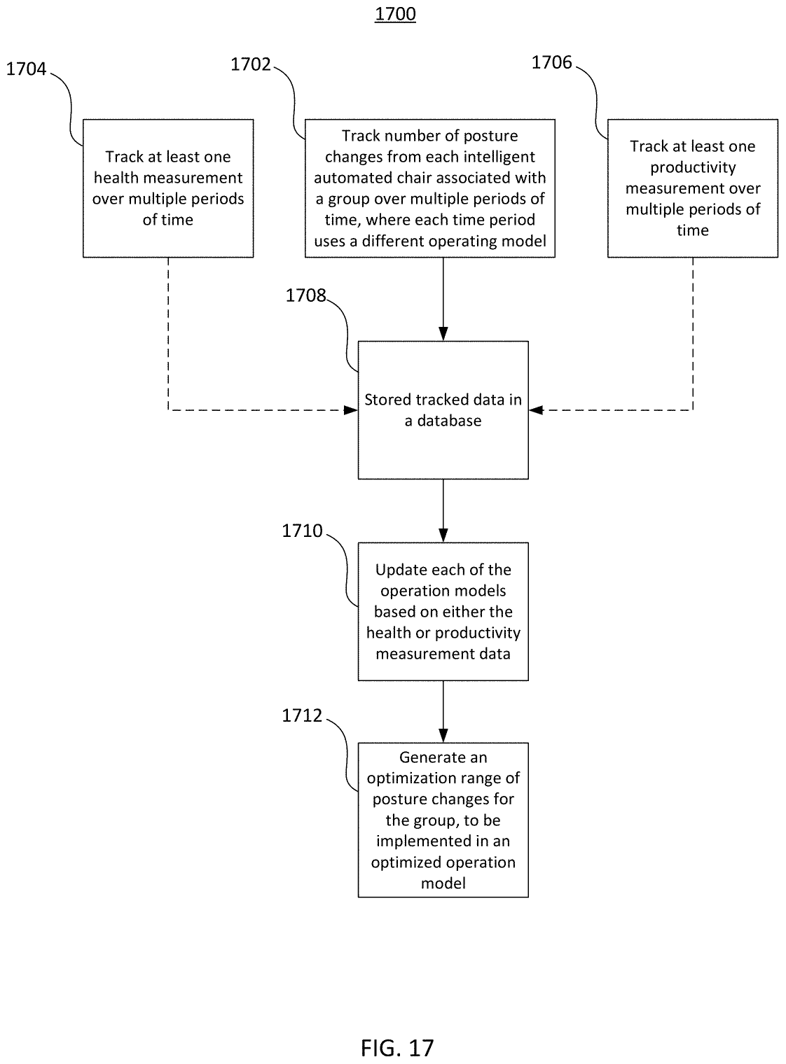

FIG. 17 illustrates a workflow 1700 for determining an optimization range for the number of posture changes for a given group. The workflow begins by tracking at least one health measurement over multiple periods of time in step 1704 or at least one productivity measurement over multiple periods of time in step 1706 along with tracking the number of posture changes over each period of time in step 1702, where the operating model is different for each period of time. All of this information is stored in a tracked database as part of step 1708. The operating models can be updated based on the tracked health data, productivity data or both in step 1710. This updating can occur begin each new period or it can be performed after several periods of differing operating models are used and then updated. Once step 1710 is completed an optimization range of posture changes for the group can be generated during step 1712 and implemented as part of the optimized operation model to be utilized by at least a subset of the group. As noted above, the optimization posture model can vary based on the specific metrics and weights given to those metrics and which metrics are trying to be influenced the most.

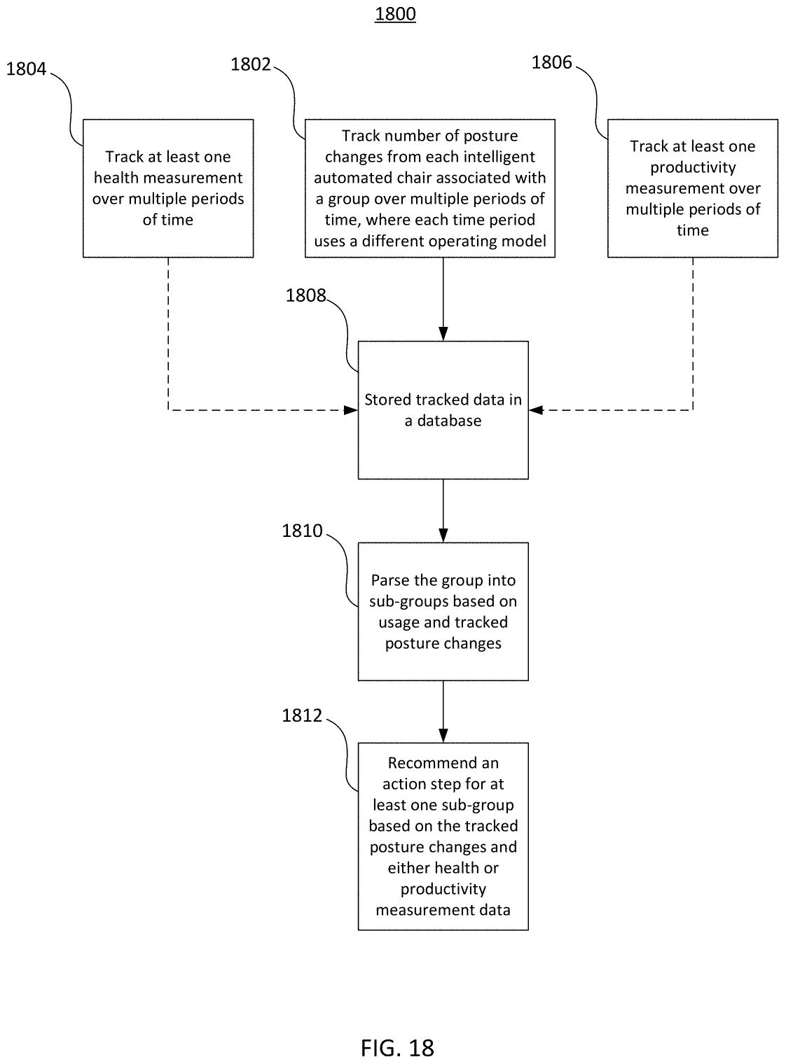

FIG. 18 illustrates a workflow 1800 for parsing a group of intelligent automated chair users into subgroups and recommending an action step to at least one of the subgroups. Similar to workflow 1700, steps 1804, 1806, 1802 and 1808 are similar to those of steps 1704, 1706, 1702 and 1708. However, in workflow 1800 once the information is tracked and stored in step 1808 a different step of parsing the group into subgroups in step 1810 can be performed. As part of the tracked posture data for the group, it can include tracked posture data for each intelligent automated chair, as well as the users using each intelligent automated chair. For example, there be a scenario where multiple shifts are run and a first employee uses an intelligent automated chair during the first shift and a second employee (or user) uses the same intelligent automated chair during a second shift. Once these subgroups are generated based on usage and tracked posture change data, the system can generate a recommended action step 1812 to be implemented for at least one of the subgroups, which can be based on the tracked posture changes and either tracked health or productivity measurement data.

These action steps can include any of the following: automated chair training program, nutritional training program, mental health training program, free medical treatment program, bonus consideration, rewards program, advancement consideration, skills training program, and increased vacation program. This list is not meant to be exhaustive, but exemplary and those skilled in the art would understand how to expand this list. The principles being conveyed are that the action steps are based on tracked posture and either tracked health measurement data, tracked productivity measurement data, or both. To further convey this, if subset group has a number of posture changes or percentage of posture changes versus time of chair usage then this could be an indicator that they might not fully understand how to operate the intelligent automated chair and thus additional training is required. In another scenario, the low usage could be an indicator of underlying health issue for which a free medical screening could help resolve. It could also be related to a productivity issue, where a training program could also be helpful. Alternatively, a ‘super’ subgroup could be identified where productivity has increased as a result of their usage, thus prompting a recommendation of providing a bonus, or advancement opportunities, or other rewards to reinforce the positive behavior. Thus, so long as the recommended action step is related to posture changes or usage of the intelligent automated chair it falls within the scope and principles being conveyed herein.

It should be understood that workflows described in FIGS. 16A-18 can be implemented using the intelligent automated chairs to implement the operating models, and track some of the data including posture changing and usage data, sensors connected to the chairs, or not can be used to track additional metrics related to health or productivity, which can be also be relayed to the databases, and cloud-based computing running weighting algorithms can be used to analyze and generate the various outputs or updates described, such as forecasts, optimization ranges, updated operating models and so forth. Intermediate mobile computing devices can also be included as part of the system components to implement these workflows, which run applications that can track or relay the various data. The cloud-based computing systems are known to have at least one processor, memory and operating instructions configured to accomplish and perform the various processes described including utilizing learning algorithms, weighted algorithms, which can be user influenced or determined, to name a few examples.

It should also be understood that the term group could also be any subgroup. For example, a design and manufacturing company may have multiple types of working conditions. There could be a group of office employees, who primarily deal with accounting matters, sales matters, administrative or management matters. There could be another group of employees that is involved in the manufacturing of a particular product or products, and even a third group that is involved with testing or experimental processes. Each group might interact differently with their automated intelligent chairs and thus different operating models for each group could be generated. This group or subgroup information can also be used as part of group profile information that can be used to determine a recommended posture change operating model.

Though much of the above has been discussed tracking the number of posture changes, it is also within the scope of these embodiments to track the type of chair postures that used most often, the duration of each, the order of which they are used. These position configurations include sitting, left leg standing, right leg standing, both legs standing, both legs standing and leaning against the back of the chair, standing with right leg up on foot rest, and standing with left leg up on foot rest. Thus, an optimization module or determination can also be focused on or include the type of positions employed, duration of each (and at various times of the day e.g. longer durations in morning and shorter in afternoon), as well as the cycling through and order of those position changes. This information can also be included as part of the overall usage information that is included.

The usage information can also be integrated into a maintenance operations prediction schedule. As with most sets of equipment, maintenance for high-use equipment or replacement can be forecasted in based on the usage of each chair and scheduled in appropriately.

From the above embodiments, some of the many advantages of these methods and systems include improving health, improving productivity, both of which generally have a direct economic impact for a company, and doing so in a minimally invasive manner, as the intelligent automated chairs naturally remind and train the users to shift postures in a way that is less disruptive and optimized for them.

While tracking posture change information for a group, it is important to do so in a manner where data privacy laws are maintained. Therefore, in some instances, any identifying information that might target or reveal privacy information associated with an individual can be anonymized.

These aspects of the invention are not meant to be exclusive and other features, aspects, and advantages of the present invention will be readily apparent to those of ordinary skill in the art when read in conjunction with the following description, appended claims, and accompanying drawings. Further, it will be appreciated that any of the various features, structures, steps, or other aspects discussed herein are for purposes of illustration only, any of which can be applied in any combination with any such features as discussed in alternative embodiments, as appropriate.

While the principles of the invention have been described herein, it is to be understood by those skilled in the art that this description is made only by way of example and not as a limitation as to the scope of the invention. Other embodiments are contemplated within the scope of the present invention in addition to the exemplary embodiments shown and described herein. Modifications and substitutions by one of ordinary skill in the art are considered to be within the scope of the present invention. Additionally, any features, structures, components, method steps which are discussed in reference to any one of the aforementioned embodiments are readily adaptable for use into and with any features of the other alternative embodiments discussed therein, with the understanding that one of ordinary skill in the art will be capable of assessing the ability of the various embodiments disclosed and be capable of making such adaptations.

1. A method of determining the number of posture changes for a group over a period of time comprising the steps of:
providing in a given area a plurality of automated chairs, each automated chair comprising:
a base portion,
a vertical support extending from the base portion,
a horizontal support interfacing with the vertical support,
a right leaf extending from the horizontal support and configured to be driven by a right motor that causes the right leaf to alter between positions of horizontal and vertical,
a left leaf extending from the horizontal support and configured to be driven by a left motor that causes the left leaf to alter between positions of horizontal and vertical, and
an automated control assembly electrically coupled to the right motor and the left motor, and configured to operate according to an automated shifting pattern that causes the right leaf and left leaf to change positions, wherein each position change is associated with a posture change, and wherein the automated control assembly is also configured to receive user input that can modify the automated shifting pattern;
tracking a number of posture changes associated with each of the plurality of automated chairs over the period of time based on the position changes; and
receiving into a database the tracked number of posture changes associated with each of the plurality of automated chairs over the period of time.

2. The method of determining the number of posture changes for a group over a period of time of claim 1, further comprising the step of tracking over the same period of time a productivity measurement.

3. The method of determining the number of posture changes for a group over a period of time of claim 2, wherein the productivity measurement includes one of the of the following: sales, customer support metrics, periodic reviews, periodic ratings, project completion timelines, work quality, attrition rate, and production output.

4. The method of determining the number of posture changes for a group over a period of time of claim 1, further comprising the step of tracking over the same period of time a health measurement.

5. The method of determining the number of posture changes for a group over a period of time of claim 4, wherein the health measurement includes one of the of the following: number of sick days, costs spent on medical care, types of medical care needed, number of health claims, number of work-related injuries, type of work-related injuries, costs spent on prescriptions, blood test measures, heart rate, body weight, amount of coffee consumed, and amount of water consumed.

6. The method of determining the number of posture changes for a group over a period of time of claim 2, further comprising the step of comparing the productivity measurement to the tracked number of posture changes.

7. The method of determining the number of posture changes for a group over a period of time of claim 6, further comprising the step of recommending a modification to the number of posture changes to be achieved by the group over a new period of time.

8. The method of determining the number of posture changes for a group over a period of time of claim 4, further comprising the step of comparing the health measurement to the tracked posture change.

9. The method of determining the number of posture changes for a group over a period of time of claim 8, further comprising the step of recommending a modification to the number of posture changes to be achieved by the group over a new period of time.

10. The method of determining the number of posture changes for a group over a period of time of claim 7, further comprising the step of updating the automated shifting pattern for at least a subset of the automated chairs based on the recommended modification to the number of posture changes to be achieved by the group over the new period of time.

11. The method of determining the number of posture changes for a group over a period of time of claim 9, further comprising the step of updating the automated shifting pattern for at least a subset of the automated chairs based on the recommended modification to the number of posture changes to be achieved by the group over the new period of time.

12. The method of determining the number of posture changes for a group over a period of time of claim 1, further includes an intelligent analysis module associated with the plurality of automated chairs that is configured to update the automated shifting pattern for at least a subset of automated chairs based on health data or productivity data associated with the group from a control period of time compared to health data or productivity data associated with the group from the period of time.

13. The method of determining the number of posture changes for a group over a period of time of claim 1, wherein the automated chair includes one or more sensors, and wherein the one or more sensors are configured to detect data that that is used to determine a productivity or health measurement.

14. The method of determining the number of posture changes for a group over a period of time of claim 1, further including an intelligent analysis module that performs the step of: parsing the group into at least two sub-groups based on the tracked number of posture changes associated with each of the plurality of automated chairs.

15. The method of determining the number of posture changes for a group over a period of time of claim 14, further comprising the step of recommending an action step for at least one of the sub-groups based on tracked posture changes associated with the at least one subgroup.

16. The method of determining the number of posture changes for a group over a period of time of claim 15, wherein the action step can be to consider providing one of the following: automated chair training program, nutritional training program, mental health training program, free medical treatment program, bonus consideration, rewards program, advancement consideration, skills training program, and increased vacation program.

17. (canceled)

18. (canceled)

19. (canceled)

20. The method of determining the number of posture changes for a group over a period of time of claim 1, wherein the automated shifting pattern includes at least one of the following parameters: sequences of positions, durations between each position change, varying durations throughout the day, and varying durations specific to each position.

21. The method of determining the number of posture changes for a group over a period of time of claim 1, further comprising the steps of:
tracking over the same period of time a productivity measurement;
tracking over the same period of time a health measurement; and
generating an updated automated shifting pattern based on the tracked productivity and health measurements.

22. (canceled)

23. The method of determining the number of posture changes for a group over a period of time of claim 1, further comprising the steps of:
tracking health measurement or productivity measurement data along with tracked posture data over several periods of time, wherein the automated shifting pattern is modified between each period to target a different range of posture changes; and
determining an optimized range of posture changes for the group.

24. The method of determining the number of posture changes for a group over a period of time of claim 23, further comprising the step of:
implementing into in an optimization operation model that guides the automated shifting pattern the determined optimized range of posture changes.

25. The method of determining the number of posture changes for a group over a period of time of claim 1, wherein the user can modify the automated shifting pattern in at least one of the following of manners: altering the frequency of the posture changes, altering the type of posture changes used, altering the posture change pattern, pausing the automated chair from continuing through the current automated shifting pattern, altering the duration of time between changes for one or more posture positions, altering the duration of time between changes for one or more periods of time.

26. An intelligent automated chair system comprising:
an automated chair comprising:
a base portion;
a vertical support extending from the base portion;
a horizontal support interfacing the vertical support;
a right leaf, configured to be driven by a motor in response to an input to alter between positions of horizontal and vertical;
a left leaf, configured to be driven by a motor in response to an input to alter between positions of horizontal and vertical; and
an automated control assembly electrically coupled to the right leaf and the left leaf, and configured to receive an updatable operating model based on compared tracked health or productivity data from a first period of time and a second period of time, wherein during the first period of time the automated chair was not used and during the second period of time the automated chair was used.

27. (canceled)

28. (canceled)

29. (canceled)

30. (canceled)

31. (canceled)

32. (canceled)

33. (canceled)

34. (canceled)

35. (canceled)

36. (canceled)
